(19) United States
(12) Patent Application Publication
(10) Pub. No.: US 2009/0275493 A1
(43) Pub. Date: Nov. 5, 2009

(54) Novel Alkaline Protease from Bacillus Gibsonii and Washing and Cleaning Agents containing said Novel Alkaline Protease

(75) Inventors: Petra Siegert, Haan (DE); Susanne Wieland, Zons/Dormagen (DE); Julia Engelskirchen, Dormagen (DE); Marion Merkel, Koeln (DE); Karl-Heinz Maurer, Erkrath (DE); Cornelius Bessler, Duesseldorf (DE)

Correspondence Address: Ratner Prestia, P.O. Box 980, Valley Forge, PA, 19482, US

(73) Assignee: Henkel AG & Co. KGaA, Duesseldorf (DE)

(21) Appl. No.: 12/502,459

(22) Filed: Jul. 14, 2009

Related U.S. Application Data
(63) Continuation of application No. PCT/EP2007/063346, filed on Dec. 5, 2007.

(30) Foreign Application Priority Data
Jan. 16, 2007 (DE) 10 2007 003 143.4

(51) Int. Cl. C11D 3/386; C07H 21/04; C12P 19/34; C12N 15/63; C12N 9/54; C12N 1/21; C12N 1/19

(52) U.S. Cl. 510/320; 536/23.2; 435/91.2; 435/320.1; 435/221; 435/252.3; 435/254.11; 510/392

(57) ABSTRACT

A novel subtilisin-type alkaline protease from Bacillus gibsonii and to sufficiently related proteins and the derivatives thereof. Also, washing and cleaning agents comprising the novel subtilisin-type alkaline protease, sufficiently related proteins and the derivatives thereof, corresponding washing and cleaning methods and their use in washing and cleaning agents, and other possible technical uses.

### CROSS REFERENCE TO RELATED APPLICATIONS

This application is a continuation under 35 U.S.C. §§ 120 and 365(c) of International Application PCT/EP2007/063346, filed on Dec. 5, 2007. This application also claims priority under 35 U.S.C. § 119 of DE 10 2007 003 143.4, filed on Jan. 16, 2007. The disclosures of PCT/EP2007/063346 and DE 10 2007 003 143.4 are incorporated herein by reference in their entirety.

### FIELD

The present invention relates to a novel subtilisin-type alkaline protease from Bacillus gibsonii and to sufficiently related proteins and derivatives thereof. It also relates to laundry detergents and cleaning agents containing these novel subtilisin-type alkaline proteases and to sufficiently related proteins and derivatives thereof, to corresponding laundry and cleaning processes and their use in laundry detergents and cleaning agents and to other possible industrial uses.

### BACKGROUND

Enzymes are established constituents of laundry detergents and cleaning agents. In this regard, proteins decompose protein-containing stains on the product to be cleaned, such as for example textiles or hard surfaces. At best, synergistic effects result between the enzymes and the usual ingredients of the agent in question. The development of laundry detergent proteases is based on naturally, preferably microbially formed enzymes. These are optimised for use in laundry detergents and cleaning agents by means of mutagenesis processes known per se, for example point mutagenesis, deletion, insertion or fusion with other proteins or protein fragments or by other modifications. Among the laundry detergent and cleaning agent proteases, subtilisins stand out due to their favourable enzymatic properties such as stability or pH optimum.

Proteases of the subtilisin type (subtilases, subtilopeptidases, EC 3.4.21.62), in particular subtilisins, are classified as serine proteases, owing to the catalytically active amino acids. They are naturally produced and secreted by microorganisms, in particular by Bacillus species. They act as unspecific endopeptidases, i.e. they hydrolyze any acid amide bonds located inside peptides or proteins. Their pH optimum is usually within the distinctly alkaline range. A review of this family is provided, for example, by the paper “Subtilases: Subtilisin-like Proteases” by R. Siezen, pages 75-95 in “Subtilisin enzymes”, edited by R. Bott and C. Betzel, New York, 1996. Subtilisins are suitable for a multiplicity of possible technical uses, as components of cosmetics and, in particular, as active ingredients of washing or cleaning agents.

The most important subtilisins and the most important strategies for their technical development are stated below.

Subtilisin BPN′, which is derived from Bacillus amyloliquefaciens and B. subtilis, respectively, has been disclosed in the studies by Vasantha et al. (1984) in J. Bacteriol., volume 159, pp. 811-819 and by J. A. Wells et al. (1983) in Nucleic Acids Research, volume 11, pp. 7911-7925. Subtilisin BPN′ serves as the reference enzyme of the subtilisins, in particular with respect to numbering of positions.

The protease subtilisin Carlsberg is presented in the publications of E. L. Smith et al. (1968) in J. Biol. Chem., volume 243, p. 2184-2191, and of Jacobs et al. (1985) in Nucl. Acids Res., volume 13, pp. 8913-8926. It is formed naturally from Bacillus licheniformis and is obtainable under the trade name Maxatase® from Genencor International Inc., Rochester, N.Y., USA, as well as under the trade name Alcalase® from Novozymes A/S, Bagsværd, Denmark.

Subtilisins 147 and 309 are commercialized under the trade names Esperase® and Savinase® by the Novozymes company. They are originally derived from Bacillus strains disclosed by the application GB 1243784.

Subtilisin DY has originally been described by Nedkov et al. 1985 in Biol. Chem. Hoppe-Seyler, Volume 366, pp. 421-430.

Further proteases of the subtilisin type, which have been isolated from Bacillus strains, are described in the more recent patent applications WO03/054185 and WO03/054184 as well as in the not yet published patent application DE 102006022216.

One strategy for enhancing the washing performance of subtilisins is to randomly or specifically substitute individual amino acids by others in the known molecules, and to test the variants obtained for their washing performance contributions. The allergenicity of the enzymes can also be improved with certain amino acid exchanges or deletions.

In order to enhance the washing performance of subtilisins, numerous patent applications pursued the strategy of inserting additional amino acids into the active loops. This strategy should be applicable in principle to all subtilisins belonging to either of the subgroups I-S1 (true subtilisins) or I-52 (highly alkaline subtilisins).

Another strategy for enhancing the performance is to modify the surface charges and/or the isoelectric point of the molecules, thereby altering their interactions with the substrate. In addition, point mutations with reduced pH-dependent variation in the molecular charge have been described. A method also based on this principle was for identifying variants that are supposedly suitable for usage in laundry detergents and cleaning agents; thus, all disclosed variants have at least one substitution at position 103. Variants are frequently described in the literature with a substitution at position 103, sometimes combined with a multiplicity of other possible substitutions. It is also possible to increase the hydrophobicity of the molecules for the purpose of enhancing the performance in laundry detergent and cleaning agents, and this may influence the stability of the enzyme.

Another method for modulating the performance of proteases is to form fusion proteins. Thus, fusion proteins composed of proteases and an inhibitor such as the Streptomyces subtilisin inhibitor are disclosed in the literature. Another possibility is, for example to couple to the cellulose binding domain (CBD), which is derived from cellulases, so as to increase the concentration of active enzyme in the direct vicinity of the substrate or to reduce the allergenicity or immunogenicity by coupling a peptide linker, and polymers thereon.

Methods for producing statistical amino acid exchanges can be based on the phage display. A modern direction in enzyme development is to combine, via statistical methods, elements from known proteins related to one another to give novel enzymes having properties that have not been achieved previously. Methods of this kind are also classified by the generic term recombination. They include, for example, the following methods: the StEP method (Zhao et al. (1998), Nat. Biotechnol., volume 16, pp. 258-261), random priming recombination (Shao et al., (1998), Nucleic Acids Res., volume 26, pp. 681-683), DNA shuffling (Semmer, W. P. C. (1994), Nature, volume 370, pp. 389-391) or recursive sequence recombination (RSR; WO 98/27230, WO 97/20078, WO 95/22625) or the RACHITT (Coco, W. M. et al. (2001), Nat. Biotechnol., volume 19, pp. 354-359). A survey of such methods is also provided by the prior article “Gerichtete Evolution und Biokatalyse” by Powell et al. (2001), Angew. Chem., vol. 113, pages 4068-4080.

Another, in particular complementary strategy, is to increase the stability of the proteases concerned and thus to increase their efficacy. For example, stabilization via coupling to a polymer has been described for proteases used in cosmetics; an enhanced skin compatibility was achieved in this way. On the other hand, especially for laundry detergents and cleaning agents, stabilizations by point mutations are more common. Thus, it is possible to stabilize proteases, particularly also in regard to their use at higher temperatures, by exchanging particular tyrosine groups with other groups. Other possible examples of stabilization via point mutagenesis, which have been described, are:
the exchange of proline for certain amino acid groups;
the introduction of polar or charged groups on the surface of the molecule;
enhancing the binding of metal ions, in particular via mutagenesis of calcium binding sites;
blocking autolysis by modification or mutagenesis;
determining the relevant positions for stabilization by an analysis of the three-dimensional structure.

It is known that proteases may be used together with α-amylases and other laundry detergent enzymes, especially lipases, in order to enhance the laundry or cleaning performance. Likewise, the use of proteases and other active substances, such as for example bleaching agents or soil release agents, in washing products is known to the person skilled in the art.

It is also known that some proteases established for use in laundry detergents are also suitable for cosmetic purposes or for the organic chemical synthesis.

The diverse technical areas of use presented here by way of example require proteases with different properties relating for example to the reaction conditions, the stability or the substrate specificity. Conversely, the possibilities of technical applications of proteases, for example in the context of a laundry detergent or cleaning product formulation, depend on additional factors such as stability of the enzyme towards high temperatures, towards oxidizing agents, its denaturing by surfactants, on folding effects or from desired synergies with other ingredients.

Thus, there continues to be a great need for industrially applicable proteases, which, owing to the large number of their application fields, in their totality cover a wide range of properties, including very subtle differences in performance.

The basis for this is expanded by novel proteases, which in turn can be further developed and targeted at specific areas of application.

### BRIEF DESCRIPTION OF THE DRAWINGS

FIG. 1 shows an alignment of the amino acid sequences of the inventive protease from Bacillus gibsonii with the most similar known subtilisins, each in the mature, i.e. processed form.

The following numbers stand for the following proteases:
1 Protease according to the invention
2 Subtilisin HP302 from Bacillus gibsonii (described in DE102006022216)
3 Subtilisin TI-1 from Bacillus gibsonii (described in WO03/054184)
4 Subtilisin TII-5 from Bacillus gibsonii (described in WO03/054185)

FIG. 2 shows the expression vector pAWA22, derived from pBC16, and possessing a promoter from B. licheniformis (PromPLi) and upstream there from a BcI I-restriction cutting site (see example 2 and Bernhard et al. (1978), 1. Bacteriol., 133 (2), pp. 897-903).

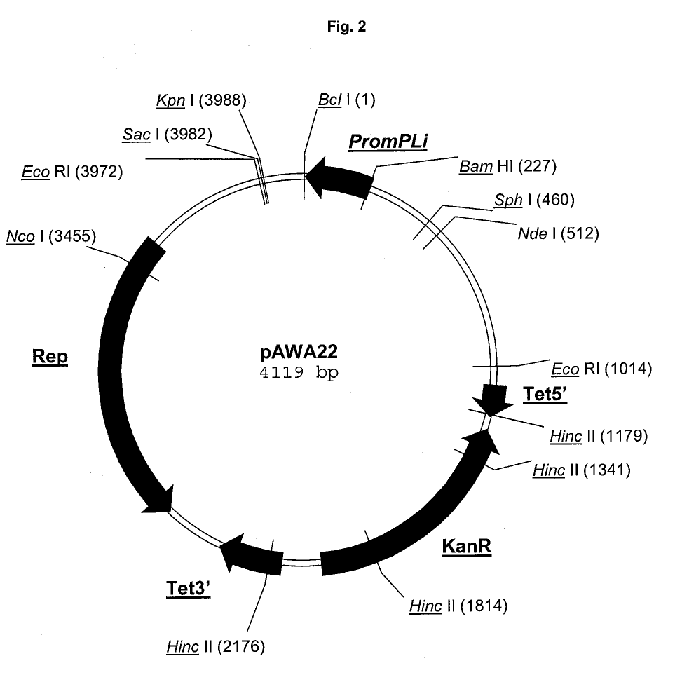

### DETAILED DESCRIPTION

Accordingly, the object of the present invention was based on finding another, not yet known protease. It was intended that the wild-type enzyme should preferably be characterized in that when used in an appropriate product it at least comes close to the enzymes established for that purpose. Of particular interest in this connection was the contribution to the performance of a laundry detergent or cleaning agent.

Further objects of the present invention can relate to the provision of proteases, especially of the subtilisin-type, which, in comparison with the prior art, exhibit improved stability towards temperature influences, pH variations, denaturing or oxidizing agents, proteolytic degradation, high temperatures, acidic or alkaline conditions or towards a change in the redox ratios. Further objects can be regarded as a reduced immunogenicity or reduced allergenic effect.

Another particular object of the present invention was to find proteases that at temperatures of 20 to 60° C. exhibit a good laundry power, preferably an improved laundry power in comparison with the proteases disclosed in the prior art, in particular those of the subtilisin type.

Another particular object of the present invention was to find proteases that in regard to the known homologous proteases from the prior art exhibit an improved laundry power in regard to at least one stain, preferably in regard to more stains.

Additional subsidiary objects consisted in the provision of nucleic acids that code for these types of proteases, and the provision of vectors, host cells and manufacturing processes that can be utilized for the production of such proteases. In addition, it was the intention to provide suitable agents, especially laundry detergents and cleaning agents, suitable laundry and cleaning processes as well as suitable end-use applications for these types of proteases. Finally, industrial application possibilities for the discovered proteases should be defined.

The object is achieved by alkaline proteases of the subtilisin type having amino acid sequences that are at least 97.5% identical to the amino acid sequence indicated from position 115 to 383 in the sequence listing under SEQ ID NO. 2 and/or differ by at most 6 amino acid positions in regard to this amino acid sequence.

Increasingly preferred are those with an increasing degree of identity with the novel alkaline protease from Bacillus gibsonii, i.e. those that only differ in 5, 4, 3 or 2 amino acid positions, and quite particularly preferably the alkali protease from Bacillus gibsonii itself.

Further solutions to the object or to the subsidiary objects and therefore to each of the individual subjects of the invention consist in nucleic acids, whose sequences are sufficiently similar to the nucleotide sequences given in SEQ ID NO. 1 and/or which code for inventive proteases, in corresponding vectors, cells, or host cells and manufacturing processes. In addition, suitable agents, especially laundry detergents and cleaning agents, suitable laundry and cleaning processes as well as suitable end-use applications for these types of proteases will be provided. Finally, industrial application possibilities for the discovered proteases will be defined.

The patent applications WO03/054185 and WO03/054184 as well as the still unpublished patent application DE 102006022216 are regarded as the closest prior art, in which is described the use of very highly homologous enzymes in laundry detergents and cleaning agents.

The naturally formed subtilisin-type alkaline protease, on which the present invention is based, as can be understood from the examples, is obtained from the culture supernatant of a novel Bacillus gibsonii strain that has been identified as such by the DSMZ (Deutsche Sammlung von Mikroorganismen und Zellkulturen). This strain was not deposited. Instead of that, for the purposes of reproducibility according to the Budapest Treaty, a plasmid comprising the nucleic acid sequence of the inventive enzyme was deposited in the DSMZ (Deutsche Sammlung für Mikroorganismen und Zellkulturen, Braunschweig) with the deposit number DSM 18912.

The present patent application followed the strategy of finding a protease-producing microorganism in a natural habitat and thus a naturally produced enzyme that satisfies as completely as possible the stated requirements.

It was possible to find such an enzyme, as described in the examples of the present application, in the form of the alkaline protease from Bacillus gibsonii.

The nucleotide sequence of the novel alkaline protease from Bacillus gibsonii is indicated in the sequence listing of the present application under SEQ ID No. 1. It comprises 1152 bp. The derived amino acid sequence is listed in SEQ ID NO. 2. It includes 383 amino acids followed by a stop codon. The first 114 amino acids thereof are probably not present in the mature protein, so that the envisaged length of the mature protein is 269 amino acids.

These sequences were compared with the protease sequences obtainable from generally accessible databases Swiss-Prot (Geneva Bioinformatics (GeneBio) S.A., Geneva, Switzerland; http://www.genebio.com/sprot.html) and GenBank (National Center for Biotechnology Information NCBI, National Institutes of Health, Bethesda, Md., USA), in order to determine the proteins with the largest homology.

The measure of homology is a percentage rate of the identity, as can be determined for example according to the methods given by D. J. Lipman and W. R. Pearson in Science 227 (1985), pp. 1435-1441. This result can refer to the whole protein or to each of the attributable regions. A further broad homology term, the similarity, also factors into the evaluation conserved variations, i.e. amino acids with similar chemical activity, because these execute mostly similar chemical activities inside the protein. For nucleic acids, only the percentage rate of identity is known.

At the DNA level, the following three genes were identified as the most similar for the complete gene: (1.) Subtilisin HP302 from Bacillus gibsonii (described in DE 102006022216) with 88% identity, (2.) Subtilisin TI-1 from Bacillus gibsonii (described in WO03/054184) with 88% identity, (3.) Subtilisin TII-5 from Bacillus gibsonii (described in WO03/054185) with 86% identity.

At the level of the DNA coding for the mature protein, the following three genes were identified as the most similar for the complete gene: (1.) Subtilisin HP302 from Bacillus gibsonii (described in DE 102006022216) with 87% identity, (2.) Subtilisin TI-1 from Bacillus gibsonii (described in WO03/054184) with 86% identity, (3.) Subtilisin TII-5 from Bacillus gibsonii (described in WO03/054185) with 84% identity.

At the level of the DNA coding for the propeptide, the following three genes were identified as the most similar for the complete gene: (1.) TII-5 from Bacillus gibsonii (described in DE WO03/054185) with 92% identity, (2.) TI-1 from Bacillus gibsonii (described in WO03/054184) with 91% identity, (3.) HP302 from Bacillus gibsonii (described in DE 102006022216) with 89% identity.

At the level of the DNA coding for the signal peptide, the following three genes were identified as the most similar for the complete gene: (1.) TII-5 from Bacillus gibsonii (described in WO03/054185) with 98% identity, (2.) HP302 from: Bacillus gibsonii (described in DE 102006022216) with 95% identity, (3.) TI-1 from Bacillus gibsonii (described in WO03/054184) with 94% identity.

At the amino acid level, the following were identified as the most similar for the total preproprotein: (1.) Subtilisin HP302 from Bacillus gibsonii (described in DE 102006022216) with 96% identity, (2.) Subtilisin TI-1 from Bacillus gibsonii (described in WO03/054184) with 95% identity, (3.) Subtilisin TII-5 from Bacillus gibsonii (described in WO03/054185) with 92% identity.

At the amino acid level, the following were identified as the most similar for the mature protein: (1.) Subtilisin HP302 from Bacillus gibsonii (described in DE 102006022216) with 97% identity, (2.) Subtilisin TI-1 from Bacillus gibsonii (described in WO03/054184) with 96% identity, (3.) Subtilisin TII-5 from Bacillus gibsonii (described in WO03/054185) with 91% identity.

At the level of the amino acids, the following were identified as the most similar for the propeptide: (1.) TII-5 from Bacillus gibsonii (described in DE WO03/054185) with 93% identity, (2.) HP302 from Bacillus gibsonii (described in DE 102006022216) with 91% identity, (3.) TI-1 from Bacillus gibsonii (described in WO03/054184) with 90% identity.

Because of the recognizable agreements and the relationship to the other cited subtilisins, this alkaline protease is to be regarded as a subtilisin.

Consequently, a subject of the present invention is any polypeptide, in particular any hydrolase, principally any subtilisin-type alkaline protease with an amino acid sequence that is identical to at least 96.5% to the amino acid sequence listed in SEQ ID NO. 2, and/or differs in at most 14 amino acid positions in regard to the amino acid sequence cited in SEQ ID NO. 2.

Among those that are increasingly preferred are those polypeptides, whose amino acid sequence is at least 97% or 97.5%, particularly preferably at least 98% or 98.5%, above all 99% or 99.5% identical to the amino acid sequences listed in SEQ ID NO. 2, and/or those, whose amino acid positions differ in at most 13, 12, 11, 10, 9, 8 or 7, in particular in at most 6, 5, 4, 3 or 2 amino acid positions, particularly preferably in one amino acid position, in regard to the amino acid sequence cited in SEQ ID NO. 2. A protein with an amino acid sequence of SEQ ID NO. 2 is quite particularly preferred.

This is because it is to be expected that the properties thereof are increasingly similar to those of the alkaline protease from B. gibsonii.

As already mentioned, on the basis of a comparison of the N-terminal sequences, the amino acids 1 to 114 are presumably to be regarded as the leader peptide, wherein the amino acids 1 to 27 presumably represent the signal peptide, and the mature protein is envisaged to extend from positions 115 to 383 according to SEQ ID No. 2. Position 384 is accordingly occupied by a stop codon and thus actually does not correspond to an amino acid. However, since the information about the end of a coding region can be regarded as an important component of an amino acid sequence, this position is included according to the invention in the region corresponding to the mature protein.

Consequently, another subject of the present invention is any polypeptide, in particular any hydrolase, principally any subtilisin-type alkaline protease with an amino acid sequence that is identical to at least 97.5% to the amino acid sequence from position 115 to position 383 listed in SEQ ID NO. 2, and/or differs in at most 6 amino acid positions in regard to this amino acid sequence.

Among those that are increasingly preferred are those polypeptides, whose amino acid sequence is at least 97% or 97.5%, particularly preferably at least 98% or 98.5%, above all 99% or 99.5% identical to the amino acid sequences from position 115 to position 383 listed in SEQ ID NO. 2, and/or those, whose amino acid positions differs in at most 5 or 4, particularly in 3 or 2 amino acid positions, particularly preferably in one amino acid position, in regard to the amino acid sequence from position 115 to position 383 cited in SEQ ID NO. 2. A protein with an amino acid sequence from position 115 to position 383 of SEQ ID NO. 2 is quite particularly preferred.

Should it emerge, for example through a N-terminal sequencing of the proteolytic protein released in vivo by Bacillus gibsonii, that the cleavage site is located not between the 114^{th} and the 115^{th} amino acid according to SEQ ID No. 2, but elsewhere, in this case these statements relate to the actual cleavage site or to the actual mature protein.

Another subject matter of the present invention also concerns fragments, particularly of the mature protein, in so far as they are novel in regard to the prior art.

Accordingly, a further subject matter of the present invention also concerns polypeptides that include an amino acid sequence with at least 81, preferably at least 90, 100 or 120, particularly preferably at least 150, 175 or 200, above all at least 225 or 250 consecutive amino acids of the amino acid sequence listed in the SEQ ID NO. 2, especially of the amino acid sequence from position 115 to 383 according to SEQ ID NO. 2.

Accordingly, a further subject matter of the present invention also concerns polypeptides that include an amino acid sequence with at least 127, preferably at least 140, 160 or 170, particularly preferably at least 180, 190 or 200, above all at least 220, 240 or 250 consecutive amino acids of the amino acid sequence listed in the SEQ ID NO. 2, especially of the amino acid sequence from position 115 to 383 according to SEQ ID NO. 2 or at most differ in one amino acid position there from.

Accordingly, a further subject matter of the present invention also concerns polypeptides that include an amino acid sequence with at least 171, preferably at least 180, 190 or 200, particularly preferably at least 210, 220 or 230, above all at least 240, 250 or 260 consecutive amino acids of the amino acid sequence listed in the SEQ ID NO. 2, especially of the amino acid sequence from position 115 to 383 according to SEQ ID NO. 2 or at most differ in two amino acid positions, preferably at most in one amino acid position there from.

Accordingly, a further subject matter of the present invention also concerns polypeptides that include an amino acid sequence with at least 192, preferably at least 200, 210 or 220, particularly preferably at least 230, 240 or 250 consecutive amino acids of the amino acid sequence listed in the SEQ ID NO. 2, especially of the amino acid sequence from position 115 to 383 according to SEQ ID NO. 2 or at most differ in three, preferably at most in two, particularly preferably at most in one amino acid position there from.

Accordingly, a further subject matter of the present invention also concerns polypeptides that include an amino acid sequence from 164 to position 382 of the amino acid sequence listed in the SEQ ID NO. 2 or however differ in at most six or five, preferably at most in four or three, particularly preferably in two positions, above all in one position there from.

As the signal peptide and the propeptide also represent moieties that as such are of inventive interest, another subject matter of the present invention concerns those peptides that are homologous to these peptides in so far as they are novel. As already mentioned, the amino acids 1 to 114 are presumably to be regarded as the leader peptide, wherein the amino acids 1 to 27 probably represent the signal peptide and correspondingly the amino acids 28 to 114 represent the propeptide. Accordingly, another subject matter of the present invention concerns polypeptides with an amino acid sequence from position 1 to 114 as well as from position 28 to 114 according to SEQ ID NO. 2 as well as peptides that differ from these amino acid sequences in at most 4, preferably in at most 3 or 2 amino acid positions, above all in exactly one amino acid position.

Another subject matter of the present invention concerns polypeptides that are coded from the inventive polynucleotides listed further below.

Increasingly preferred among these are those polypeptides that are derived from a nucleotide sequence which is as similar as possible to the nucleotide sequence indicated in SEQ ID No. 1, in particular over the partial region corresponding to positions 115 to 384 of the polypeptide in SEQ ID No. 2.

This is because it is to be expected that these nucleic acids code for proteins whose properties are increasingly similar to those of the inventive alkaline protease from Bacillus gibsonii, especially of the mature protein. In this case too, as for all following embodiments, it is again true that these statements relate to the actual mature protein should it emerge that the cleavage site of the protein is located elsewhere than indicated above.

In view of the closest prior art, namely in view of the proteases subtilisin HP302 from Bacillus gibsonii (described in DE 102006022216), subtilisin TI-1 from Bacillus gibsonii (described in WO03/054184) and subtilisin TII-5 from Bacillus gibsonii (described in WO03/054185), a subject matter of the present invention preferably concerns those inventive polypeptides that exhibit, in comparison with this closest prior art, an improved laundry performance in regard to at least one stain, preferably in regard to at least two stains, in particular selected from grass on cotton, milk/oil on cotton, whole egg/carbon black on cotton, chocolate milk/carbon black on cotton and blood/milk on cotton, particularly at a wash temperature of 20 to 60° C., preferably 30° C., and preferably when used in a liquid laundry detergent. An improved laundry performance at 30° C. when used in a liquid laundry detergent in regard to all of the abovementioned stains is quite particularly preferred.

The inventively most preferred embodiment is all alkaline proteases of the subtilisin-type, whose amino acid sequence is identical to the amino acid sequence listed in SEQ ID NO. 2 as a whole, preferably to the positions 115 to 383 and/or whose amino acid sequence can be derived from the nucleotide sequence listed in SEQ ID NO. 1, preferably from the positions 343 to 1152.

The alkaline proteases from Bacillus gibsonii that are newly discovered and provided by the present application are those of this type.

This is a protease that is not yet known in the prior art. It can be isolated, manufactured and utilized, as listed in the examples. As is also documented in the examples, it is further characterized in that when used in an appropriate agent, its activity at least approximates or even exceeds that of the enzymes established for this purpose.

The inventive polypeptides preferably concern enzymes, particularly preferably hydrolases, especially proteases, particularly preferably endopeptidases, above all proteases of the subtilisin type, or fragments thereof. Consequently, the inventive polypeptides are preferably capable of hydrolyzing acid amide bonds of proteins, especially those located internally in the proteins. The fragments of the polypeptides can concern in particular protein domains that can be suitable for example for producing functional chimeric enzymes.

For the development of industrial proteases that in particular are applicable in detergents, it can serve, as a natural microbially produced enzyme, as a starting point to be optimized for the desired application by means of mutagenetic methods that are known per se, for example point mutagenesis, fragmentation, deletion, insertion or fusion with other proteins or protein fragments or by other modifications. These types of optimizations can be for example adaptations to the effects of temperature, pH fluctuations, redox conditions and/or other influences that are relevant to the industrial field of use. Examples are an improvement in the resistance to oxidation, in the stability towards denaturing agents or proteolytic degradation, towards high temperatures, acidic or strongly alkaline conditions, a change in the sensitivity towards calcium ions or other cofactors, and a reduction in the immunogenicity or allergenic effect.

It is possible for this purpose to alter by targeted point mutations the surface charges or the loops involved in catalysis or substrate binding for example. A starting point for this is an alignment with known proteases. This makes it possible for positions to be discovered that by their variation are able to produce optional improvements of the properties of the protein.

The mutagenesis processes involve the associated nucleotide sequence that is listed in SEQ ID NO. 1 or the sufficiently similar nucleotide sequences that are illustrated below as a separate inventive subject matter. Suitable molecular biological methods are described in the prior art, for example in pertinent handbooks such as that by Fritsch, Sambrook und Maniatis “Molecular cloning: a laboratory manual”, Cold Spring Harbour Laboratory Press, New York, 1989.

Accordingly, further embodiments of the present invention are, in addition to the already inventively mentioned protein variants based on point mutation or substitution mutation, also all polypeptides derived from the previously mentioned inventive polypeptides, especially from polypeptides with an amino acid sequence according to SEQ ID NO. 2 or from position 115 to position 383 according to SEQ ID NO. 2, by insertion mutagenesis and/or by substitution mutagenesis and/or inversion mutagenesis and/or by fusion with at least one other protein or protein fragment, in particular those peptides with insertions and/or deletions and/or inversions of up to 50 amino acids, particularly preferably of up to 40, 30 or 20, especially of up to 15, 10 or 5, above all of up to 4, 3 or 2 amino acids, above all with deletions and/or insertions of exactly one amino acid.

Thus for example, it is possible to delete individual amino acids on the termini or in the loops of the enzyme, without losing the proteolytic activity. Mutations of this type are described for example in WO 99/49057 A1. WO 01/07575 A2 teaches that the allergenicity of the proteases in question can be reduced by such deletions and therefore overall their applicability can be improved. Fragmentation benefits the later described aspect of insertion mutagenesis or substitution mutagenesis and/or fusion with other enzymes. In regard to the intended use of these enzymes, it is particularly preferred when they also possess a proteolytic activity after fragmentation or deletion mutagenesis.

Numerous documents from the prior art also disclose advantageous effects of insertions and substitutions in subtilases; among these are also the publications WO 99/49057 and WO 01/07575. In principal, besides the substitution of individual amino acids, this also includes the substitution of a plurality of contiguous amino acids. This also includes novel combinations of larger enzyme segments such as the above-cited fragments with other proteases or proteins of another function. Therefore it is possible, for example in accordance with WO 99/57254, to equip an inventive protein or fragment thereof through peptidic linkers or directly as the fusion protein with binding domains from other proteins, for example the cellulose binding domains, and thereby to more effectively design the hydrolysis of the substrate. Similarly, inventive proteins can also be linked for example with amylases or cellulases so as to execute a dual function.

Among the inventive polypeptides, those protein variants are preferred which possess one or more amino acid exchanges in the positions 3, 4, 36, 42, 47, 56, 61, 69, 87, 96, 99, 101, 102, 104, 114, 118, 120, 130, 139, 141, 142, 154, 157, 188, 193, 199, 205, 211, 224, 229, 236, 237, 242, 243, 255 and 268 in the numbering of the alkaline protease from Bacillus lentus.

Inventive chimeric proteins possess in the broadest sense a proteolytic activity. This can be executed or modified by a part of a molecule that derives from an inventive polypeptide. The chimeric proteins may thus also be located over their entire length outside the region claimed above. The sense of such a fusion consists in, for example, providing or modifying a certain function or partial function with the help of the fused-on inventive protein part. It is irrelevant in the context of the present invention whether such a chimeric protein consists of a single polypeptide chain or a plurality of sub-units. The latter alternative can be effected for example post-translationally or first after a purification step by means of a targeted proteolytic cleavage by breaking down a single chimeric polypeptide chain into several.

Thus, for example, it is possible, based on WO 99/57254, to provide a protein of the invention or parts thereof via peptide linkers or directly as fusion protein with binding domains from other proteins, for example the cellulose binding domain, and thus to make hydrolysis of the substrate more efficient. Such a binding domain might also originate from a protease, for example in order to enhance the binding of the protein of the invention to a protease substrate. This increases the local protease concentration, which may be advantageous in individual applications, for example in the treatment of raw materials. Similarly, inventive proteins can also be linked for example with amylases or cellulases so as to execute a dual function.

The inventive polypeptides that can be obtained by insertion mutation are assigned to the inventive chimeric proteins due to their fundamental similarity. Substitution variations also belong here, i.e. those in which single regions of the molecule have been substituted with elements from other proteins.

The significance of insertion and substitution mutagenesis is as in hybrid formation, to combine individual properties, functions or partial functions of inventive proteins with those of other proteins. This also includes a shuffling or novel combination of partial sequences from various proteases to obtained variants. In this way proteins can be obtained that beforehand had not yet been described. Such techniques enable drastic effects down to very subtle modulations in activity.

They include, for example, the following methods: the StEP method (Zhao et al. (1998), Nat. Biotechnol., volume 16, pp. 258-261), random priming recombination (Shao et al., (1998), Nucleic Acids Res., volume 26, pp. 681-683), DNA shuffling (Semmer, W P. C. (1994), Nature, volume 370, pp. 389-391) or recursive sequence recombination (RSR; WO 98/27230, WO 97/20078, WO 95/22625) or the RACHITT method (Coco, W. M. et al. (2001), Nat. Biotechnol., volume 19, pp. 354-359). Such processes are necessarily coupled with a selection or screening process subsequent to the mutagenesis and expression, so as to recognize variants having the desired properties. As these techniques apply to the DNA level, the starting point for the biotechnological production is made available with each of the associated newly produced genes.

Inversion mutagenesis, meaning a partial reversal of the sequence, can be regarded as a special form of both deletion as well as of insertion. Such variants can likewise be targeted or randomly produced.

Preference is given to all inventive polypeptides mentioned to date and which are characterized in that they are able per se to hydrolyze protein.

Such entities are categorized according to the official Enzyme Nomenclature 1992 of the IUBMB under 3.4 (peptidases). Among these, preference is given to endopeptidases, particularly of the groups 3.4.21 serine proteinases, 3.4.22 cysteine proteinases, 3.4.23 aspartate proteinases and 3.4.24 metallo proteinases. Of these, serine proteinases (3.4.21) are particularly preferred, and among these subtilases and, among these, very particularly subtilisins (compare “Subtilases: Subtilisin-like proteases” by R. Siezen, pages 75-95 in “Subtilisin enzymes”, edited by R. Bott and C. Betzel, New York, 1996). Among these in turn, preference is given to subtilisins of the group IS-2, the highly alkaline subtilisins.

In this connection, active molecules are preferred to inactive ones, because in particular the proteolysis that is performed is important for example in the areas of use detailed below.

The above listed fragments also possess, in the broadest sense, a proteolytic activity, for example for complexing a substrate or for forming a structural element required for the hydrolysis. They are preferred when they can themselves be employed for the hydrolysis of another protein without the need for further protease components to be present. This relates to the activity, which can be performed by a protease per se; the presence, which may be necessary at the same time, of buffer substances, cofactors, etc. remains unaffected by this.

An interaction of different molecular parts for the hydrolysis naturally exists in deletion mutants rather than in fragments and ensues in particular in fusion proteins, quite particularly those that emanate from a shuffling of related proteins. Where this results in maintenance, modification, specification or else first attainment of a proteolytic function in the widest sense, the deletion variants and the fusion proteins are proteins of the invention. Preferred representatives of this subject of the invention among these are those able per se to hydrolyze a protein substrate without the need for further protease components to be present.

A preferred embodiment is represented by all proteins, protein fragments or fusion proteins mentioned to date which are characterized in that they are additionally stabilized.

In this way their stability during storage and/or during their use, for example during the washing process, is increased such that their activity lasts longer and is consequently boosted. Coupling to polymers, for example, can increase the stability of inventive proteases. This requires that prior to use in suitable agents, the proteins be bonded with such polymers by means of a coupling step.

Stabilizations that are possible by point mutagenesis of the molecule itself are preferred. No further process steps would then be required after having extracted the protein. Some point mutations that are suitable for this are known from the prior art. Thus, proteins can be stabilized for example by exchanging certain tyrosine moieties with others.

Other possibilities are for example:
the exchange of proline for certain amino acid groups;
the introduction of polar or charged groups on the surface of the molecule;
modifying the binding of metal ions, in particular of calcium binding sites.
according to U.S. Pat. No. 5,453,372, proteins can be protected against the influence of denaturing agents such as surfactants by certain mutations on the surface.

Another possibility for stabilizing against increased temperature and the effect of surfactants can reside in the stabilization by the exchange of amino acids in close proximity to the N-terminus with those that come into contact with the remainder of the molecule through non-covalent interactions and consequently contribute to maintaining the globular structure.

A preferred embodiment is represented by all inventive polypeptides mentioned to date and which are characterized in that they are additionally derivatized.

Derivatives are understood to mean those proteins that are derived from the listed proteins by an additional modification. These types of modifications can influence for example the stability, substrate specificity or the binding strength to the substrate or the enzymatic activity. They can also serve to reduce the allergenicity and/or immunogenicity of the protein and thereby increase its skin compatibility, for example.

Such derivatizations can be effected biologically, for example by the produced host organism in connection with the protein biosynthesis. Here, couplings of low molecular weight compounds such as lipids or oligosaccharides are particularly emphasized.

However, derivatizations can also be effected chemically, for example by the chemical transformation of a side chain or by the covalent bonding of another, for example macromolecular compound onto the protein. For example, the coupling of amines on carboxylic groups of an enzyme is thus possible in order to change the isoelectric point. Moreover, macromolecules, such as proteins, for example can be bonded through e.g. bifunctional chemical compounds to inventive proteins. Such a macromolecule can be a binding domain, for example. These types of derivatives are particularly suitable for use in washing or cleaning agents. Analogously, protease inhibitors can also be bonded through linkers, especially amino acid linkers, to the inventive proteins. Couplings with other macromolecular compounds, such as polyethylene glycol, improve the molecule in regard to further properties, such as stability or skin compatibility.

In the broadest sense, derivatives of inventive proteins can also be understood to mean preparations of these enzymes. Depending on extraction, work up or preparation, a protein can be blended with various other materials, for example from the cultures produced by microorganisms. Certain other materials can also be purposely added to a protein, for example to increase its storage stability. Therefore, all preparations of an inventive protein are also in accordance with the invention. This is also independent of whether this enzymatic activity is actually displayed by a specific preparation. It may be desired that it possesses no or only limited activity during storage, and first develops its proteolytic function at the time of use. This can be controlled for example by suitable accompanying substances such as for example protease inhibitors.

A preferred embodiment is represented by all proteins, protein fragments, fusion proteins or derivatives, which are characterized in that they have at least one antigenic determinant in common with one of the above described inventive polypeptides.

The secondary structural elements of a protein and its three dimensional folding are decisive for the enzymatic activities. Thus, domains that significantly differ from each other in their primary structure can form spatially largely conformable structures and therefore make possible the same enzymatic behavior. Such commonalities in the secondary structure are usually identified as autologous antigenic determinants of antiserums or of pure or monoclonal antibodies. Similar proteins or derivatives can be detected and classified in this way by means of immunochemical cross reactions. Consequently, such proteins that may possibly not be classified by their degree of homology in the primary structure but arguably by their immunochemical affinity to the above defined inventive proteins, protein fragments, fusion proteins or derivatives are also precisely included in the scope of protection of the present invention.

A preferred embodiment is illustrated by all those inventive polypeptides that have been listed up to now, which are characterized in that they are obtained from a natural source, in particular from a microorganism.

For example they can be single cell fungi or bacteria. Mostly they can be more easily extracted and handled than the multicellular organisms or the cell cultures derived from metazoa; although these can represent reasonable options for specific embodiments and are thus not fundamentally excluded from the subject of the invention.

It is possible that naturally occurring products can indeed manufacture an inventive enzyme; however under the investigated conditions this only expresses to a limited extent and/or releases into the surrounding medium. However, this does not rule out suitable environmental conditions or other factors from being experimentally determined and that their application could stimulate a commercially reasonable production of the inventive protein. Such a regulation mechanism can be purposely employed for biotechnological production. If this is also not possible then they can still be used for isolating the associated gene.

Among these, those from gram-positive bacteria are particularly preferred. This is because they do not possess an external membrane and thus immediately release secreted proteins into the surrounding medium.

Those from gram-positive bacteria of the genus Bacillus are quite particularly preferred.

A priori, Bacillus proteases possess favorable characteristics for various fields of industrial application. They include a certain stability towards increased temperature, oxidizing or denaturing agents. In addition, most experience has been obtained with microbial enzymes in regard to their biotechnological production, for example concerning the construction of cost-effective cloning vectors, the selection of host cells and growth conditions or the estimation of risk, such as for example the allergenicity. Furthermore, bacilli are established as production organisms having a particularly high production performance in industrial processes. The wealth of experience acquired for the manufacture and use of these proteases is of great benefit to the inventive further development of these enzymes. This concerns for example their compatibility with other chemical compounds, such as, for example, the ingredients of washing or cleaning agents.

Among those of the Bacillus species, once again those from the species Bacillus gibsonii, especially from the inventively used strain of Bacillus gibsonii, are preferred.

This is because the embodiment of the inventive enzymes was originally obtained from it. Its associated sequences are given in the sequence transcript. The above-described variants can be manufactured from it or from related strains by the use of standard microbiological methods, such as, for example PCR and/or the known point mutagenesis methods.

The nucleic acids that serve to accomplish the invention represent a further solution to the problem of the invention and thereby a separate subject matter of the invention.

Using today's generally known methods, such as for example chemical synthesis or the polymerase chain reaction (PCR) in combination with molecular biological and/or protein chemical standard methods, it is possible for the person skilled in the art to manufacture the complete genes with the help of known DNA sequences and/or amino acid sequences. These types of methods are known, for example from the “Lexikon der Biochemie”, Spektrum Akademischer Verlag, Berlin, 1999, volume 1, pp. 267-271 and Volume 2, pp. 227-229. In particular, this is possible if one can revert to a strain deposited in a collection of strains. For example, with PCR primers, which can be synthesized by means of a known sequence, and/or through isolated mRNA molecules, the gene in question can be synthesized from such strains, cloned and optionally further treated, for example mutagenized.

Nucleic acids form the starting point for virtually all molecular biological investigations and developments as well as the production of proteins. This includes in particular the gene sequencing and the deduction of the associated sequence of amino acids, each type of mutagenesis (see above) and the protein expression.

Mutagenesis for the development of proteins having defined characteristics is also called “protein engineering”. Examples of characteristics that are optimised have already been described above. Such a mutagenesis can be targeted or carried out with random methods, for example with a screening and selection method directed to the final activity of the cloned genes, for example by hybridisation with nucleic acid sensors, or on the gene products, the proteins, for example regarding their activity. Further development of the inventive proteases can be organized according to the considerations presented in the publication “Protein engineering” by P. N. Bryan (2000) in Biochim. Biophys. Acta, volume 1543, pp. 203-222.

Accordingly, a further subject matter of the present invention also concerns polynucleotides that code for inventive polypeptides, in particular hydrolases, especially alkaline proteases of the subtilisin type. Accordingly, a subject matter of the present invention is especially polynucleotides selected from the group consisting of:
a) polynucleotide with a nucleic acid sequence according to SEQ ID NO: 1,
b) polynucleotide with a nucleic acid sequence from position 1 to 342 according to SEQ ID NO: 1,
c) polynucleotide with a nucleic acid sequence from position 1 to 81 according to SEQ ID NO: 1,
d) polynucleotide with a nucleic acid sequence from position 82 to 342 according to SEQ ID NO: 1,
e) polynucleotide with a nucleic acid sequence from position 343 to 1152 according to SEQ ID NO: 1,
f) polynucleotide coding for a polypeptide with an amino acid sequence according to SEQ ID NO: 2,
g) polynucleotide coding for a polypeptide with an amino acid sequence from position 1 to 114 according to SEQ ID NO: 2,
h) polynucleotide coding for a polypeptide with an amino acid sequence from position 28 to 114 according to SEQ ID NO: 2,
i) polynucleotide coding for a polypeptide with an amino acid sequence from position 115 to 383 according to SEQ ID NO: 2,
j) polynucleotide coding for an inventive polypeptide,
k) naturally occurring or synthetically produced mutants or polymorphic forms or alleles of a polynucleotides according to (a) or (e) containing up to 80, preferably up to 50, 45, 40, 35 or 30, especially up to 25, 20, 15 or 10, above all up to 9, 8, 7, 6, 5, 4, 3 or 2 mutations, quite particularly preferably containing exactly one mutation,
l) naturally occurring or synthetically produced mutants or polymorphic forms or alleles of a polynucleotides according to (b) or (d) containing up to 25, preferably up to 22, 20, 18, 16 or 15, particularly preferably up to 14, 13, 12, 11 or 10, above all up to 9, 8, 7, 6, 5, 4, 3 or 2 mutations, quite particularly preferably containing exactly one mutation,
m) polynucleotides with a sequence homology or identity of at least 90%, preferably at least 91, 92, 93, 94 or 95%, particularly preferably at least 96, 97, 98 or 99%, with respect to a polynucleotide according to (a) or (e),
n) polynucleotides with a sequence homology or identity of at least 93%, preferably at least 94, 95 or 96%, particularly preferably at least 97, 98 or 99%, with respect to a polynucleotide according to (d),
o) polynucleotides hybridizing under stringent conditions with a polynucleotide according to (a) to (i), wherein, “under stringent conditions” is preferably understood to mean incubation at 60° C. in a solution comprising 0.1×SSC and 0.1% sodium dodecylsulfate (SDS), wherein 20×SSC designates a solution comprising 3 M sodium chloride and 0.3 M sodium citrate (pH 7.0),
p) polynucleotides consisting of at least 200, preferably at least 250, 300, 350 or 400, particularly preferably at least 450, 500, 550 or 600, above all at least 650, 700, 750 or 800 sequential nucleic acids of a polynucleotide according to (a), (b), (d), (e), (f), (g), (h) or (i),
q) polynucleotides containing deletions and/or insertions and/or inversions of up to 50, preferably up to 40, 30 or 20, particularly preferably up to 15, 10 or 5, especially up to 4, 3 or 2 nucleotides, above all insertions and/or deletions of exactly one nucleotide with respect to a polynucleotide according to (a) to (p), especially with respect to a polynucleotide according to (a) or (e),
r) polynucleotides comprising at least one of the polynucleotides cited under (a) to (q),
s) polynucleotides complementary to polynucleotides according to (a) to (r).

The polynucleotides can exist as a single strand or as a double strand. Beside the deoxyribonucleic acids, an inventive subject matter is also the homologous and complementary ribonucleic acids.

A subject matter of the present invention also particularly concerns those polynucleotides, in which, by taking into account the differentiating codon usage, certain regions of a host organism held responsible for the expression are replaced by other regions, so as to enable the expression of the inventive polypeptide.

In accordance with the abovementioned statements, the following are increasingly preferred among the above-described inventive nucleic acids:
those that are obtained from a natural source, in particular from a microorganism;
among the above, those wherein the microorganism concerns a gram-positive bacterium;
among the above, those wherein the gram-positive bacterium concerns one of the genus Bacillus; and
among the above, those wherein the Bacillus species concerns Bacillus gibsonii, in particular the inventively used strain.

A separate subject matter of the invention is formed by vectors that comprise one of the previously identified, inventive nucleic acid regions, especially one that codes for one of the previously identified polypeptides.

In order to deal with the relevant inventive nucleic acids, and therefore in particular to prepare the production of inventive polypeptides, said acids are suitably ligated in vectors. Such vectors and the associated working methods are extensively described in the prior art. A great number and a broad variation of vectors are commercially available, both for cloning as well as for expression. These include for example vectors that are derived from bacterial plasmids, bacteriophages or viruses, or predominantly synthetic vectors. Furthermore, they are differentiated according to the nature of the cell types, in which they are capable of establishing themselves, for example according to vectors for gram-negative, for gram-positive bacteria, for yeasts or for higher eukaryotes. They form suitable starting points for molecular biological and biochemical investigations, for example, as well as for the expression of the gene in question or associated proteins.

In one embodiment the inventive vectors concern cloning vectors.

In addition to the storage, the biological amplification or the selection of the gene of interest, the cloning vectors are suitable for its molecular biological characterization. At the same time they represent transportable and storable forms of the claimed nucleic acids and are also starting points for molecular biological techniques that are not linked with cells, such as for example PCR or in vitro mutagenesis processes.

Preferably, the inventive vectors are expression vectors.

Such expression vectors are the basis for the realization of the corresponding nucleic acids in biological production systems and hence for the production of the associated proteins. Preferred embodiments of this subject matter of the invention are expression vectors that carry genetic elements required for expression, for example the natural localizing promoter originating before the gene or a promoter from another organism. These elements can be arranged in the form of a so-called expression cassette, for example. Alternatively, individual or all regulation elements can also be prepared from the relevant host cell. The expression vectors are particularly preferably matched in regard to further characteristics, such as, for example the optimum copy number, the chosen expression system, especially the host cells (see below).

In addition, it is advantageous for a high expression rate if the expression vector comprises preferably only the gene in question as the insert and no larger 5′- or 3′-non coding regions. Such inserts are obtained for example if the fragment obtained after statistical treatment of the chromosomal DNA of the starting strain with a restriction enzyme after the sequencing has been purposely cut once more before the integration into the expression vector.

Vector pAWA22 is an example of an expression vector. Further vectors from the prior art are available to the person skilled in the art and a great many are commercially available.

Cells, which, after genetic modification, comprise an inventive polynucleotide, form a separate subject matter of the invention.

These cells comprise the genetic information for the synthesis of an inventive protein. Among these, in contrast to the abovementioned, likewise claimed natural producers, are meant in particular those cells that have been provided with the nucleic acids according to the invention by methods known per se, or which are derived from such cells. The host cells suitably selected for this purpose are those, which can be cultured relatively easily and/or provide high product yields.

They enable for example the amplification of the corresponding gene, but also its mutagenesis or transcription and translation and finally the biotechnological production of the protein in question. This genetic information can be integrated either extrachromosomally as the single genetic element, i.e. for bacteria present in the plasmidic localization or be integrated into a chromosome. The choice of a suitable system depends on the issues, such as for example the nature and period of storage of the gene, or of the organism or the nature of the mutagenesis or selection. Thus, in the prior art for example are described mutagenetic and selection methods based on bacteriophages—and their specific host cells—for the development of laundry detergent enzymes.

The inventive polynucleotide is preferably part of one of the above-mentioned inventive vectors, especially a cloning or expression vector.

In this way they are relevant to the realization of the present invention.

In addition, those cells are preferred that express and preferably secrete an inventive polypeptide.

The host cells that form the proteins enable their biotechnological production. In principle, all organisms, i.e. prokaryotes, eukaryotes or cyanophytae are suitable host cells for protein expression. Those host cells are preferred, which can be genetically handled with ease, for example in relation to the transformation with the expression factor and its stable establishment and the regulation of the expression, for example single cell fungi or bacteria. In addition, preferred host cells are those with a good microbiological and biotechnological handleability. For example this relates to ease of cultivation, high growth rates, low demands on fermentation media and good production rates and secretion rates for foreign proteins. Laboratory strains that are geared to expression are preferably selected. They are commercially available or can be obtained from generally accessible collections of strains. Theoretically, each inventive protein can be obtained in this way from a plurality of host organisms. The optimum expression system for the individual case must be experimentally determined from the abundance of different systems available from the prior art.

Host cells are particularly preferred that are themselves protease-negative and hence do not degrade cultured proteins.

Preferred embodiments are illustrated by such host cells that, due to suitable genetic elements, can be regulated in their activity, for example by the controlled addition of chemical compounds, by changing the conditions of cultivation or as a function of the respective cell density. This controllable expression makes possible a very economical production of the proteins of interest. Suitably the gene, expression vector and host cell are matched to one another, for example in regard to the genetic elements required for expression (ribosome binding site, promoters, terminators) or the codon usage.

Preferred among these are those expression hosts that secrete the cultured protein into the surrounding medium, as the protein can be relatively easily recovered.

Moreover, host cells, which are bacteria, are preferred.

Bacteria are characterized by short generation times and low demands on the cultivation conditions. In this manner, cost effective processes can be established. Moreover, there exists an extensive wealth of experience with bacteria in fermentation technology. Gram-negative or Gram-positive bacteria may be suitable for a specific production for a wide variety of reasons, which are to be ascertained experimentally for each individual case, such as nutrient sources, product formation rate, time required etc.

A preferred embodiment involves a Gram-negative bacterium, in particular one of the genera Escherichia coli, Klebsiella, Pseudomonas or Xanthomonas, in particular strains of E. coli K12, E. coli B or Klebsiella planticola, and very especially derivatives of the strain Escherichia coli BL21 (DE3), E. coli RV308, E. coli DH5α, E. coli JM109, E. coli XL-1 or Klebsiella planticola (Rf).

This is because a large number of proteins are secreted into the periplasmic space with Gram-negative bacteria such as, for example, E. coli. This can be advantageous for specific applications. The application WO 01/81597 A1 discloses a method, which achieves expulsion of the expressed proteins by Gram-negative bacteria as well. Such a system is also suitable for manufacturing inventive proteins. The Gram-negative bacteria mentioned as preferred are usually easily available, i.e. commercially or from public collections of strains, and can be optimized for specific preparation conditions in association with other components such as, for instance, vectors, which are likewise available in large numbers.

An alternative, not less preferred embodiment, involves a Gram-positive bacterium, in particular one of the genera Bacillus, Staphylococcus or Corynebacterium, quite particularly of the species Bacillus lentus, B. licheniformis, B. amyloliquefaciens, B. subtilis, B. globigii, B. gibsonii, B. pumilus or B. alcalophilus, Staphylococcus carnosus or Corynebacterium glutamicum.

This is because Gram-positive bacteria have the fundamental difference from Gram-negative ones of immediately releasing secreted proteins into the nutrient medium which surrounds the cells and from which if desired the expressed proteins of the invention can be directly purified from the nutrient medium. In addition, they are related or identical to most of the organisms of origin of industrially important subtilisins and mostly themselves produce comparable subtilisins, so that they have a similar codon usage and their protein synthesis apparatus is naturally appropriately configured. A further advantage is that with this process a mixture of inventive proteins can be obtained with the cultured subtilisins that are endogenously formed from the host strains. This type of co expression also emanates from the application WO 91/02792. When this is not required, the protease genes that are naturally present in the host cell have to be permanently or temporarily inactivated (see above).

Moreover, host cells, which are eurokaryotic cells, preferably of the genus Saccharomyces, are preferred.

Examples of these are fungi such as Actinomycetes or even yeasts such as Saccharomyces or Kluyveromyces. Thermophilic fungal expression systems are presented for example in WO 96/02653 A1. These are particularly suitable for the expression of temperature stable variants. Modifications that eukaryotic systems carry out, particularly in connection with the protein synthesis, include for example the binding of low molecular weight compounds such as membrane anchors or oligosaccharides. These types of oligosaccharide modifications can be desirable for lowering the allergenicity. A co expression with the enzymes that are naturally formed from these types of cells, such as for example cellulases, can also be advantageous.

Processes for manufacturing an inventive polypeptide represent an independent subject matter of the invention.

This includes any method for preparing a polypeptide of the invention described above, for example chemical synthetic methods.

In contrast, however, all molecular biological, microbiological or biotechnological manufacturing processes that are established in the prior art, that build on the above designated inventive nucleic acids and already discussed in detail above are preferred. For this, corresponding to the above statement, one can revert to the nucleic acids or to the correspondingly derived mutations or partial sequences thereof listed in the sequence protocol SEQ ID NO. 1.

Methods preferred in this connection are those taking place with the use of a vector designated above and particularly preferably with the use of an advantageously genetically modified cell designated above. In this way the correspondingly preferred genetic information is made available in a microbiologically exploitable form.

Embodiments of the present invention based on the associated nucleic acid sequences can also be cell-free expression systems, in which the protein biosynthesis is reconstructed in vitro. All of the elements listed above can also be combined in new processes to manufacture the proteins according to the invention. A plurality of possible combinations of process steps is conceivable for each inventive protein, such that optimum processes have to be experimentally determined for each practical single case.

In agreement with the above statements, those methods among the cited methods are preferred, in which the nucleotide sequence has been adapted in one or, preferably, more codons to the codon usage of the host strain.

Compositions that comprise a previously described polypeptide represent a separate subject matter of the invention.

All types of compositions, in particular mixtures, formulations, solutions etc., whose suitability is improved by the addition of one of the inventive proteins described above, are hereby included in the scope of protection of the present invention. Depending on the field of application, this can concern for example solid mixtures, for example powders with freeze dried or encapsulated proteins, or agents in gel or liquid form. Preferred formulations comprise for example buffer substances, stabilizers, reaction partners and/or cofactors of the proteases and/or other constituents that are synergistic with the proteases. Among these in particular are agents for the application areas listed further below. Additional application areas emerge from the prior art and are illustrated for example in the handbook “Industrial enzymes and their applications” by H. Uhlig, Wiley-Verlag, New York, 1998.

Accordingly, possible fields of application are in particular the use for obtaining or treating raw materials or intermediate products in textile manufacturing, especially for removing protective layers on fabrics, particularly on wool or silk, as well as the use for the care of textiles that comprise natural fibers, especially wool or silk.

Natural fibers in particular, such as wool or silk, for example, are distinguished by a characteristic, microscopic surface structure. Said surface structure can, in the long term, result in undesired effects such as, for example, felting, as discussed by way of example for wool in the article by R. Breier in Melliand Textilberichte from 4.1.2000 (p. 263). In order to avoid such effects, the natural raw materials are treated with agents according to the invention, which contribute, for example, to smoothing the flaked surface structure based on protein structures, and thereby counteract felting.

Accordingly, methods for treating textile raw materials and for textile care, in which the inventive polypeptides are used in at least one of the procedural steps, are also a subject matter of the invention. Among these, methods for textile raw materials, fibers or textiles containing natural constituents are preferred, especially for those containing wool or silk. This can concern processes for example in which materials are prepared for treating textiles, for example for an anti-pilling finish or for example processes that add a care component when cleaning worn textiles.

Additional possible fields of use are, for example
the use for biochemical analyses or for synthesizing low molecular weight compounds or of proteins, preferred among these being the use for determining the end groups in the context of a peptide sequence analysis;
the use for the preparation, cleaning or synthesis of natural materials or biological resources;
the use for the treatment of natural raw materials, in particular for the treatment of surfaces, very particularly in a method for the treatment of leather, especially for the depilation of leather;
the use for the treatment of photographic films, in particular for removing gelatine-containing or similar protective layers; and
the use for preparing food or animal feed, in particular for the enzymatic treatment of soya milk and/or soya milk products.

Fundamentally, the addition of the previously mentioned inventive polypeptides in all additional technical fields, for which it proves to be suitable, is hereby included in the scope of protection of the present invention.

In another possible use of the invention, the polypeptides according to the invention are used for cosmetic compositions. These are understood to include all types of cleaning and caring compositions for human skin or human hair, especially cleaning compositions. The composition, depending on the application purpose, can also be a pharmaceutical composition.

Proteases also play a crucial part in the desquamation of human skin (T. Egelrud et al., Acta Derm. Venerol., volume 71 (1991), pp. 471-474). Proteases are accordingly also used as bioactive components in skincare products in order to support degradation of the desmosome structures increasingly present in dry skin. The use of subtilisin proteases containing amino acid exchanges in the positions R99G/A/S, S154D/E and/or L211 D/E for cosmetic purposes is described for example in WO 97/07770 A1. In agreement with the above statements, the inventive proteases can be further developed through the appropriate point mutations. Proteases of the invention, in particular those whose activity is controlled, for example, after mutagenesis or due to addition of appropriate substances interacting with them, are therefore also suitable as active components in skin- or hair-cleaning compositions or care compositions. Particular preference is given to those preparations of said enzymes, which, as described above, are stabilized, for example by coupling to macromolecular supports (compare U.S. Pat. No. 5,230,891), and/or are derivatized by point mutations at highly allergenic positions such that their compatibility with human skin is increased.

As exemplary inventive cosmetics and/or pharmaceuticals, may be mentioned shampoos, soaps, washing lotions, creams, peelings as well as compositions for oral care, dental care and dental prosthesis care. In particular, these compositions may also comprise ingredients such as those mentioned below for laundry detergents and cleaning agents.

Accordingly, corresponding cosmetic cleaning and care methods and the use of proteolytic enzymes of this kind for cosmetic purposes, are also included in this subject matter of the invention, in particular in appropriate agents such as, for example, shampoos, soaps or washing lotions or in care compositions provided, for example, in the form of creams. The use in a peeling medicament or the use for its manufacture is also included in this subject matter.

A particularly preferred subject matter is illustrated by laundry detergents and cleaning agents that comprise inventive polypeptides. As is shown in the examples of the present application, laundry detergents and cleaning agents containing an inventively preferred protease surprisingly demonstrated an increased washing power compared to compositions containing conventional proteases.

In the context of the present application, “washing performance” or “cleaning performance” of a laundry detergent or cleaning agent is understood to mean the effect that the agent in question produces on the soiled article, for example textiles or objects with hard surfaces. Individual components of such agents, in particular the enzymes according to the invention, are assessed in regard to their contribution to the washing performance or cleaning performance of the laundry detergent or cleaning agent as a whole. It should be particularly borne in mind here that the enzymatic properties of an enzyme do not allow a straightforward analysis of its contribution to the washing performance of an agent. In fact, other factors play a role besides the enzymatic activity, in particular factors such as stability, substrate binding, binding onto the goods being cleaned or interactions with other ingredients of the laundry detergent or cleaning agent, particularly further possible synergies during removal of the soils.

Accordingly, another subject matter of the present invention concerns laundry detergents and cleaning agents, especially surfactant and/or bleaching agent-containing, which comprise a polypeptide according to the invention.

The inventive laundry detergents and cleaning agents can refer to all the various possible types of cleaning compositions, both concentrates and compositions to be used without dilution, for use on a commercial scale in washing machines or in hand washing or manual cleaning. These include, for example, laundry detergents for fabrics, carpets or natural fibers, for which the term “laundry detergent” is used in the present invention. These also include, for example, dishwashing detergents for dishwashing machines or manual dishwashing detergents or cleaners for hard surfaces, such as metal, glass, china, ceramic, tiles, stone, painted surfaces, plastics, wood or leather, for which the term “cleaning agent” is used in the present invention. In the broader sense, sterilization compositions and disinfectants are also to be regarded as laundry detergents and cleaning agents in the context of the invention.

Embodiments of the present invention include all types established by the prior art and/or all required usage forms of the inventive washing or cleaning agents. These include for example solid, powdered, liquid, gelled or pasty agents, optionally from a plurality of phases, compressed or non-compressed; further included are, for example: extrudates, granulates, tablets or pouches, both in bulk and also packed in portions.

In a preferred embodiment, the inventive laundry detergent or cleaning agents comprise the above described polypeptides according to the invention, in particular subtilisin-type alkaline proteases, in an amount of 2 μg to 20 mg, preferably 5 μg to 17.5 mg, particularly preferably 20 μg to 15 mg, quite particularly preferably 50 μg to 10 mg per gram of the agent. All whole numbered and non-whole numbered values between these numbers are included.

The protease activity in agents of this type can be determined according to the method described in Tenside, volume 7, (1970), pp. 125-132. Consequently, it is reported in PU (protease units).

When comparing the performances of two laundry detergent enzymes, such as for example in the examples of the present application, protein equivalent and activity equivalent addition must be differentiated. The protein equivalent addition is used particularly for genetically obtained preparations that are essentially free of side activities. This enables a declaration of whether the same amounts of protein—as a measure for the yield of the fermentation production—lead to comparable results. When the respective proportions of active substance to total protein (the value of the specific activity) differ widely, then an activity equivalent comparison is to be recommended, because in this way the respective enzymatic properties are compared. It is generally true that a low specific activity can be compensated by adding a larger amount of protein. This is ultimately an economic consideration.

In addition to an inventive polypeptide, an inventive laundry detergent or cleaning agent optionally comprises further ingredients such as additional enzymes, enzyme stabilizers, surfactants, e.g. non-ionic, anionic and/or amphoteric surfactants, and/or bleaching agents, and/or builders, as well as optional further conventional ingredients, which are described below.

Preferred non-ionic surfactants are alkoxylated, advantageously ethoxylated, particularly primary alcohols preferably containing 8 to 18 carbon atoms and, on average, 1 to 12 moles of ethylene oxide (EO) per mole of alcohol, in which the alcohol group may be linear or, preferably, methyl-branched in the 2-position or may contain linear and methyl-branched groups in the form of the mixtures typically present in oxoalcohol groups. In particular, however, alcohol ethoxylates with linear alcohol groups of natural origin with 12 to 18 carbon atoms, e.g. from coco-, palm-, tallow- or oleyl alcohol, and an average of 2 to 8 EO per mole alcohol are preferred. Exemplary preferred ethoxylated alcohols include C₁₂₋₁₄ alcohols with 3 EO or 4 EO, C₉₋₁₁ alcohols with 7 EO, C₁₃-C₁₅ alcohols with 3 EO, 5 EO, 7 EO or 8 EO, C₁₂-C₁₈-alcohols with 3 EO, 5 EO or 7 EO and mixtures thereof, such as mixtures of C₁₂-C₁₄ alcohol with 3 EO and C₁₂-C₁₈ alcohol with 5 EO. The cited degrees of ethoxylation constitute statistically average values that can be a whole or a fractional number for a specific product. Preferred alcohol ethoxylates have a narrowed homolog distribution (narrow range ethoxylates, NRE). In addition to these non-ionic surfactants, fatty alcohols with more than 12 EO can also be used. Examples of these are tallow fatty alcohol with 14 EO, 25 EO, 30 EO or 40 EO.

Another class of preferred non-ionic surfactants which may be used, either as the sole non-ionic surfactant or in combination with other non-ionic surfactants are alkoxylated, preferably ethoxylated or ethoxylated and propoxylated fatty acid alkyl esters preferably containing 1 to 4 carbon atoms in the alkyl chain, in particular fatty acid methyl esters.

A further class of non-ionic surfactants, which can be advantageously used, are the alkyl polyglycosides (APG). Suitable alkyl polyglycosides satisfy the general Formula RO(G)_{z} where R is a linear or branched, particularly 2-methyl-branched, saturated or unsaturated aliphatic group containing 8 to 22, preferably 12 to 18 carbon atoms and G is the symbol that stands for a glycose unit containing 5 or 6 carbon atoms, preferably for glucose. Here, the degree of glycosidation z is between 1.0 and 4.0, preferably between 1.0 and 2.0 and particularly between 1.1 and 1.4. Linear alkyl polyglucosides are preferably employed, i.e. alkyl polyglycosides, in which the polyglycosyl group is a glucose group and the alkyl group is an n-alkyl group.

Non-ionic surfactants of the amine oxide type, for example N-coco alkyl-N,N-dimethylamine oxide and N-tallow alkyl-N,N-dihydroxyethylamine oxide, and from the fatty acid alkanolamides may also be suitable. The quantity of these non-ionic surfactants is preferably no more than the quantity in which the ethoxylated fatty alcohols are used and, particularly no more than half that quantity.

Other suitable surfactants are polyhydroxyfatty acid amides corresponding to the Formula (I),

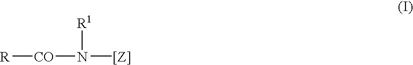

in which RCO stands for an aliphatic acyl group with 6 to 22 carbon atoms, R¹ for hydrogen, an alkyl or hydroxyalkyl group with 1 to 4 carbon atoms and [Z] for a linear or branched polyhydroxyalkyl group with 3 to 10 carbon atoms and 3 to 10 hydroxyl groups. The polyhydroxyfatty acid amides are known substances, which may normally be obtained by reductive amination of a reducing sugar with ammonia, an alkylamine or an alkanolamine and subsequent acylation with a fatty acid, a fatty acid alkyl ester or a fatty acid chloride.

The group of polyhydroxyfatty acid amides also includes compounds corresponding to the Formula (II),

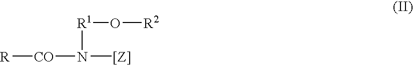

in which R stands for a linear or branched alkyl or alkenyl group containing 7 to 12 carbon atoms, R¹ for a linear, branched or cyclic alkyl group or an aryl group containing 2 to 8 carbon atoms and R² for a linear, branched or cyclic alkyl group or an aryl group or an oxyalkyl group containing 1 to 8 carbon atoms, C₁₋₄ alkyl or phenyl groups being preferred, and [Z] is a linear polyhydroxyalkyl group, of which the alkyl chain is substituted by at least two hydroxyl groups, or alkoxylated, preferably ethoxylated or propoxylated derivatives of that group.

[Z] is preferably obtained by reductive amination of a sugar, for example glucose, fructose, maltose, lactose, galactose, mannose or xylose. The N-alkoxy- or N-aryloxy-substituted compounds may then be converted into the required polyhydroxyfatty acid amides by reaction with fatty acid methyl esters in the presence of an alkoxide as catalyst.

Exemplary suitable anionic surfactants are those of the sulfonate and sulfate type. Suitable surfactants of the sulfonate type are, advantageously C₉₋₁₃ alkylbenzene sulfonates, olefin sulfonates, i.e. mixtures of alkene- and hydroxyalkane sulfonates, and disulfonates, as are obtained, for example, from C₁₂₋₁₈ monoolefins having a terminal or internal double bond, by sulfonation with gaseous sulfur trioxide and subsequent alkaline or acidic hydrolysis of the sulfonation products. Those alkane sulfonates, obtained from C₁₂₋₁₈ alkanes by sulfochlorination or sulfoxidation, for example, followed by hydrolysis or neutralization, are also suitable. The esters of α-sulfofatty acids (ester sulfonates), e.g. the α-sulfonated methyl esters of hydrogenated coco-, palm nut- or tallow acids are likewise suitable.

Further suitable anionic surfactants are sulfated fatty acid esters of glycerine. Fatty acid glycerine esters are understood to mean the mono-, di- and triesters and also mixtures of them, such as those obtained by the esterification of a monoglycerine with 1 to 3 moles fatty acid or the transesterification of triglycerides with 0.3 to 2 moles glycerine. Preferred sulfated fatty acid esters of glycerol in this case are the sulfated products of saturated fatty acids with 6 to 22 carbon atoms, for example caproic acid, caprylic acid, capric acid, myristic acid, lauric acid, palmitic acid, stearic acid or behenic acid.

Preferred alk(en)yl sulfates are the alkali metal and especially sodium salts of the sulfuric acid half-esters derived from the C₁₂-C₁₈ fatty alcohols, for example from coconut butter alcohol, tallow alcohol, lauryl, myristyl, cetyl or stearyl alcohol or from C₁₀-C₂₀ oxo alcohols and those half-esters of secondary alcohols of these chain lengths. Additionally preferred are alk(en)yl sulfates of the said chain lengths, which contain a synthetic, straight-chained alkyl group produced on a petrochemical basis and which show similar degradation behaviour to the suitable compounds based on fat chemical raw materials. The C₁₂-C₁₆ alkyl sulfates and C₁₂-C₁₅ alkyl sulfates and C₁₄-C₁₅ alkyl sulfates are preferred on the grounds of laundry performance. 2,3-Alkyl sulfates are also suitable anionic surfactants.

Sulfuric acid mono-esters derived from straight-chained or branched C₇₋₂₁ alcohols ethoxylated with 1 to 6 moles ethylene oxide are also suitable, for example 2-methyl-branched alcohols with an average of 3.5 mole ethylene oxide (EO) or C₁₂₋₁₈ fatty alcohols with 1 to 4 EO. Due to their high foaming performance, they are only used in fairly small quantities in cleaning compositions, for example in amounts of up to 5% by weight, usually from 1 to 5% by weight.

Other suitable anionic surfactants are also the salts of alkylsulfosuccinic acid, which are also referred to as sulfosuccinates or esters of sulfosuccinic acid and the monoesters and/or di-esters of sulfosuccinic acid with alcohols, preferably fatty alcohols and especially ethoxylated fatty alcohols. Preferred sulfosuccinates comprise C₈₋₁₈ fatty alcohol groups or mixtures of them. Especially preferred sulfosuccinates comprise a fatty alcohol group derived from ethoxylated fatty alcohols and may be considered as non-ionic surfactants (see description above). Once again the particularly preferred sulfosuccinates are those, whose fatty alcohol groups are derived from ethoxylated fatty alcohols with narrow range homolog distribution. It is also possible to use alk(en)ylsuccinic acids with preferably 8 to 18 carbon atoms in the alk(en)yl chain, or salts thereof.

Soaps in particular can be considered as further anionic surfactants. Saturated fatty acid soaps are suitable, such as the salts of lauric acid, myristic acid, palmitic acid, stearic acid, hydrogenated erucic acid and behenic acid, and especially soap mixtures derived from natural fatty acids such as coconut oil fatty acid, palm kernel oil fatty acid or tallow fatty acid.

Anionic surfactants, including the soaps, may be in the form of their sodium, potassium or ammonium salts or as soluble salts of organic bases, such as mono-, di- or triethanolamine. Preferably, the anionic surfactants are in the form of their sodium or potassium salts, especially in the form of the sodium salts.

The surfactants can be comprised in the inventive cleaning compositions or laundry detergents in a total amount of preferably 5 to 50 wt. %, particularly 8 to 30 wt. %, based on the finished composition.

The inventive laundry detergents or cleaning compositions can comprise bleaching agent. Among the compounds, which serve as bleaches and liberate H₂O₂ in water, sodium percarbonate, sodium perborate tetrahydrate and sodium perborate monohydrate are of particular importance. Examples of further bleaching agents that may be used are peroxypyrophosphates, citrate perhydrates and H₂O₂-liberating peracidic salts or peracids, such as persulfates or persulfuric acid. The urea peroxyhydrate percarbamide that can be described by the formula H₂N—CO—NH₂.H₂O₂ is also suitable. Particularly when agents are used to clean hard surfaces, for example in automatic dishwashers, they can, if desired, also comprise bleaching agents from the group of the organic bleaching agents, although in principal they can also be used for washing textiles. Typical organic bleaching agents are the diacyl peroxides, such as e.g. dibenzoyl peroxide. Further typical organic bleaching agents are the peroxy acids, wherein the alkylperoxy acids and the arylperoxy acids may be named as examples. Preferred representatives that can be added are peroxybenzoic acid and ring-substituted derivatives thereof, such as alkyl peroxybenzoic acids, but also peroxy-α-naphthoic acid and magnesium monoperphthalate, the aliphatic or substituted aliphatic peroxy acids, such as peroxylauric acid, peroxystearic acid, ε-phthalimidoperoxycaproic acid [phthaloiminoperoxyhexanoic acid PAP)], o-carboxybenzamidoperoxycaproic acid, N-nonenylamido peradipic acid and N-nonenylamido persuccinates and aliphatic and araliphatic peroxydicarboxylic acids, such as 1,12-diperoxycarboxylic acid, 1,9-diperoxyazelaic acid, diperoxysebacic acid, diperoxybrassylic acid, diperoxyphthalic acids, 2-decyldiperoxybutane-1,4-dioic acid, N,N-terephthaloyl-di(6-aminopercaproic acid).

The bleaching agent content of the laundry detergent or cleaning composition is preferably 1 to 40 wt. % and particularly 10 to 20 wt. %, perborate monohydrate or percarbonate being advantageously used.

The preparations can also comprise bleach activators in order to achieve an improved bleaching action for washing temperatures of 60° C. and below and particularly during the pre-treatment wash. Bleach activators, which can be used, are compounds which, under perhydrolysis conditions, yield aliphatic peroxycarboxylic acids having preferably 1 to 10 carbon atoms, in particular 2 to 4 carbon atoms, and/or optionally substituted perbenzoic acid. Substances, which carry O-acyl and/or N-acyl groups of said number of carbon atoms and/or optionally substituted benzoyl groups, are suitable. Preference is given to polyacylated alkylenediamines, in particular tetraacetyl ethylenediamine (TAED), acylated triazine derivatives, in particular 1,5-diacetyl-2,4-dioxohexahydro-1,3,5-triazine (DADHT), acylated glycolurils, in particular 1,3,4,6-tetraacetyl glycoluril (TAGU), N-acylimides, in particular N-nonanoyl succinimide (NOSI), acylated phenol sulfonates, in particular n-nonanoyl- or isononanoyloxybenzene sulfonate (n- or iso-NOBS), acylated hydroxycarboxylic acids, such as triethyl O-acetylcitrate (TEOC), carboxylic acid anhydrides, in particular phthalic anhydride, isatoic anhydride and/or succinic anhydride, carboxylic amides, such as N-methyldiacetamide, glycolide, acylated polyhydric alcohols, in particular triacetin, ethylene glycol diacetate and 2,5-diacetoxy-2,5-dihydrofuran and the enol esters known from the German Patent applications DE 196 16 693 and DE 196 16 767 and acetylated sorbitol and mannitol or their mixtures (SORMAN) described in the European Patent application EP 0 525 239, acylated sugar derivatives, in particular pentaacetyl glucose (PAG), pentaacetyl fructose, tetraacetyl xylose and octaacetyl lactose as well as acetylated, optionally N-alkylated glucamine and gluconolactone, triazole or triazole derivatives and/or particulate caprolactams and/or caprolactam derivatives, preferably N-acylated lactams, for example N-benzoyl caprolactam and N-acetyl caprolactam, which are known from the international patent applications WO 94/27970, WO 94/28102, WO 94/28103, WO 95/00626, WO 95/14759 and WO 95/17498. The hydrophilically substituted acylacetals, known from the German Patent application DE 196 16 769 and the acyllactams described in the German Patent application DE 196 16 770 as well as the international Patent application WO 95/14075 are also preferably used. The combinations of conventional bleach activators known from the German Patent application DE 44 43 177 can also be used. Nitrile derivatives such as cyanopyridines, nitrile quats, for example N-alkylammonium acetonitrile, and/or cyanamide derivatives can also be used. Preferred bleach activators are sodium 4-(octanoyloxy)benzene sulfonate, n-nonanoyl- or isononanoyloxybenzene sulfonate (n- or iso-NOBS), undecenoyloxybenzene sulfonate (UDOBS), sodium dodecanoyloxybenzene sulfonate (DOBS), decanoyloxybenzoic acid (DOBA, OBC 10) and/or dodecanoyloxybenzene sulfonate (OBS 12), and N-methylmorpholinum acetonitrile (MMA). These types of bleach activators are comprised in the usual quantity range of 0.01 to 20 wt. %, preferably in amounts of 0.1 wt. % to 15 wt. %, particularly 1 wt. % to 10 wt. %, based on the total composition.

In addition to, or instead of the conventional bleach activators mentioned above, so-called bleach catalysts may also be incorporated. These substances are bleach-boosting transition metal salts or transition metal complexes such as, for example, manganese-, iron-, cobalt-, ruthenium- or molybdenum-salen or carbonyl complexes. Manganese, iron, cobalt, ruthenium, molybdenum, titanium, vanadium and copper complexes with nitrogen-containing tripod ligands, as well as cobalt-, iron-, copper- and ruthenium-ammine complexes may also be employed as the bleach catalysts, wherein those compounds that are described in DE 197 09 284 A1 are preferably employed.

Generally, inventive washing or cleaning agents comprise one or more builders, in particular zeolites, silicates, carbonates, organic cobuilders and—where there are no ecological grounds against their use—also phosphates. The last are particularly preferred builders employed in cleaning compositions for automatic dishwashers.

Suitable silicate builders are the crystalline, layered sodium silicates corresponding to the general formula NaMSiₓO₂ₓ₊₁yH₂O, wherein M is sodium or hydrogen, x is a number from 1.6 to 4, preferably 1.9 to 4.0 and y is a number from 0 to 20, preferred values for x being 2, 3 or 4. These types of crystalline layered silicates are described, for example, in the European Patent application EP 0 164 514. Preferred crystalline layered silicates of the given formula are those in which M stands for sodium and x assumes the values 2 or 3. Both β- and also δ-sodium disilicates Na₂Si₂O₅ yH₂O are particularly preferred. These types of compounds are commercially available, for example, under the designation SKS® (Clariant). SKS-6® is mainly a δ-sodium disilicate with the formula Na₂Si₂O₅ yH₂O, and SKS-7® is mainly the α-sodium disilicate. On reaction with acids (e.g. citric acid or carbonic acid), δ-sodium silicate affords Kanemit NaHSi₂O₅ yH₂O, commercially available under the trade names SKS-9® and SKS-10® (Clariant). It can also be advantageous to chemically modify these layered silicates. The alkalinity, for example, of the layered silicates can be suitably modified. In comparison with the δ-sodium disilicate, layered silicates, doped with phosphate or carbonate, exhibit a different crystal morphology, dissolve more rapidly and show an increased calcium binding ability. Thus, layered silicates of the general formula x Na₂O y SiO₂ z P₂O₅ in which the ratio x to y corresponds to a number 0.35 to 0.6, the ratio x to z to a number from 1.75 to 1200 and the ratio y to z to a number from 4 to 2800, are described in the patent application DE 196 01 063. The solubility of the layered silicates can also be increased by employing particularly finely dispersed layered silicates. Compounds of the crystalline layered silicates with other ingredients can also be used. Compounds with cellulose derivatives, which possess advantages in the disintegration action, and which are particularly used in detergent tablets, as well as compounds with polycarboxylates, for example citric acid or polymeric polycarboxylates, for example copolymers of acrylic acid can be particularly cited in this context.

Other useful builders are amorphous sodium silicates with a modulus (Na₂O:SiO₂ ratio) of 1:2 to 1:3.3, preferably 1:2 to 1:2.8 and more preferably 1:2 to 1:2.6, which dissolve with a delay and exhibit multiple wash cycle properties. The delay in dissolution compared with conventional amorphous sodium silicates can have been obtained in various ways, for example by surface treatment, compounding, compressing/compacting or by over-drying. In the context of this invention, the term “amorphous” also means “X-ray amorphous”. In other words, the silicates do not produce any of the sharp X-ray reflexes typical of crystalline substances in X-ray diffraction experiments, but at best one or more maxima of the scattered X-radiation, which have a width of several degrees of the diffraction angle. However, particularly good builder properties may even be achieved where the silicate particles produce indistinct or even sharp diffraction maxima in electron diffraction experiments. This is to be interpreted to mean that the products have microcrystalline regions between 10 and a few hundred nm in size, values of up to at most 50 nm and especially up to at most 20 nm being preferred. Compacted/densified amorphous silicates, compounded amorphous silicates and over dried X-ray-amorphous silicates are particularly preferred.

An optionally suitable fine crystalline, synthetic zeolite containing bound water, is preferably zeolite A and/or P. Zeolite MAP® (commercial product of the Crosfield company), is particularly preferred as the zeolite P. However, zeolite X and mixtures of A, X and/or P are also suitable. Commercially available and preferably used in the context of the present invention is, for example, also a co-crystallizate of zeolite X and zeolite A (ca. 80 wt. % zeolite X), which is marketed by CONDEA Augusta S.p.A. under the trade name VEGOBOND AX® and which can be described by the Formula

nNa₂O.(1-n)K₂O.Al₂O₃.(2-2.5)SiO₂.(3.5-5.5)H₂O

Suitable zeolites have a mean particle size of less than 10 μm (volume distribution, as measured by the Coulter Counter Method) and contain preferably 18 to 220% by weight and more preferably 20 to 22% by weight of bound water.

Naturally, the generally known phosphates can also be added as builders, in so far that their use should not be avoided on ecological grounds. In the detergent and cleaning agent industry, among the many commercially available phosphates, the alkali metal phosphates are the most important and pentasodium or pentapotassium triphosphates (sodium or potassium tripolyphosphate) are particularly preferred.

“Alkali metal phosphates” is the collective term for the alkali metal (more particularly sodium and potassium) salts of the various phosphoric acids, in which metaphosphoric acids (HPO₃)ₙ and orthophosphoric acid (H₃PO₄) and representatives of higher molecular weight can be differentiated. The phosphates combine several inherent advantages: they act as alkalinity sources, prevent lime deposits on machine parts and lime incrustations in fabrics and, in addition, contribute towards the cleaning effect.

Sodium dihydrogen phosphate NaH₂PO₄ exists as the dihydrate (density 1.91 g·cm⁻³, melting point 60° C.) and as the monohydrate (density 2.04 g·cm⁻³). Both salts are white, readily water-soluble powders that on heating, lose the water of crystallization and at 200° C. are converted into the weakly acidic diphosphate (disodium hydrogen diphosphate, Na₂H₂P₂O₇) and, at higher temperatures into sodium trimetaphosphate (Na₃P₃O₉) and Maddrell's salt (see below). NaH₂PO₄ shows an acidic reaction. It is formed by adjusting phosphoric acid with sodium hydroxide to a pH value of 4.5 and spraying the resulting “mash”. Potassium dihydrogen phosphate (primary or monobasic potassium phosphate, potassium biphosphate, KDP), KH₂PO₄, is a white salt with a density of 2.33 g·cm⁻³, has a melting point of 253° C. [decomposition with formation of potassium polyphosphate (KPO₃)] and is readily soluble in water.

Disodium hydrogen phosphate (secondary sodium phosphate), Na₂HPO₄, is a colorless, very readily water-soluble crystalline salt. It exists in anhydrous form and with 2 mol (density 2.066 g·cm³, water loss at 95° C.), 7 mol (density 1.68 g·cm³, melting point 48° C. with loss of 5H₂O) and 12 mol of water (density 1.52 g·cm⁻³, melting point 35° C. with loss of 5H₂O), becomes anhydrous at 100° C. and, on fairly intensive heating, is converted into the diphosphate Na₄P₂O₇. Disodium hydrogen phosphate is prepared by neutralization of phosphoric acid with soda solution using phenolphthalein as the indicator. Dipotassium hydrogen phosphate (secondary or dibasic potassium phosphate), K₂HPO₄, is an amorphous white salt, which is readily soluble in water.

Trisodium phosphate, tertiary sodium phosphate, Na₃PO₄, consists of colorless crystals that as the dodecahydrate have a density of 1.62 g·cm⁻³ and a melting point of 73-76° C. (decomposition), as the decahydrate (corresponding to 19-20% P₂O₅) a melting point of 100° C. and in anhydrous form (corresponding to 39-40% P₂O₅) a density of 2.536 g·cm⁻³. Trisodium phosphate is readily soluble in water with an alkaline reaction and is manufactured by evaporating a solution of exactly 1 mole disodium phosphate and 1 mole NaOH. Tripotassium phosphate (tertiary or tribasic potassium phosphate), K₃PO₄, is a white deliquescent granular powder with a density of 2.56 g·cm⁻³, has a melting point of 1340° C. and is readily soluble in water through an alkaline reaction. It is produced by e.g. heating Thomas slag with carbon and potassium sulfate. Despite their higher price, the more readily soluble and therefore highly effective potassium phosphates are often preferred to corresponding sodium compounds in the detergent industry.

Tetrasodium diphosphate (sodium pyrophosphate), Na₄P2O₇, exists in anhydrous form (density 2.534 g·cm³, melting point 988° C., a figure of 880° C. has also been mentioned) and as the decahydrate (density 1.815-1.836 gcm⁻³, melting point 94° C. with loss of water). Both substances are colorless crystals that dissolve in water with an alkaline reaction. Na₄P₂O₇ is formed when disodium phosphate is heated to more than 200° C. or by reacting phosphoric acid with soda in a stoichiometric ratio and spray drying the solution. The decahydrate complexes heavy metal salts and hardness salts and, hence, reduces the hardness of water. Potassium diphosphate (potassium pyrophosphate), K₄P₂O₇, exists in the form of the trihydrate and is a colorless hygroscopic powder with a density of 2.33 g·cm⁻³, which is soluble in water, the pH of a 1% solution at 25° C. being 10.4.

Relatively high molecular weight sodium and potassium phosphates are formed by condensation of NaH₂PO₄ or KH₂PO₄. They may be divided into cyclic types, namely the sodium and potassium metaphosphates, and chain types, the sodium and potassium polyphosphates. The chain types in particular are known by various different names: fused or calcined phosphates, Graham's salt, Kurrol's salt and Maddrell's salt. All higher sodium and potassium phosphates are known collectively as condensed phosphates.

The industrially important pentasodium triphosphate, Na₅P₃O₁₀ (sodium tripolyphosphate), is anhydrous or crystallizes with 6H₂O to a non-hygroscopic, white, water-soluble salt which has the general formula NaO—[P(O)(ONa)—O]ₙ—Na where n=3. Around 17 g of the salt free from water of crystallization dissolve in 100 g of water at room temperature, around 20 g at 60° C. and around 32 g at 100° C. After heating the solution for 2 hours to 100° C., around 8% orthophosphate and 15% diphosphate are formed by hydrolysis. In the preparation of pentasodium triphosphate, phosphoric acid is reacted with soda solution or sodium hydroxide in a stoichiometric ratio and the solution is spray-dried. Similarly to Graham's salt and sodium diphosphate, pentasodium triphosphate solubilizes many insoluble metal compounds (including lime soaps, etc.). K₅P₃O₁₀ (potassium tripolyphosphate), is marketed for example in the form of a 50% by weight solution (>23% P₂O₅, 25% K₂O). The potassium polyphosphates are widely used in the laundry detergent and cleaning industry. Sodium potassium tripolyphosphates also exist and are also usable in the scope of the present invention. They are formed for example when sodium trimetaphosphate is hydrolyzed with KOH:

(NaPO₃)₃+2KOH→Na₃K₂P₃O₁₀+H₂O

According to the invention, they may be used in exactly the same way as sodium tripolyphosphate, potassium tripolyphosphate or mixtures thereof. Mixtures of sodium tripolyphosphate and sodium potassium tripolyphosphate or mixtures of potassium tripolyphosphate and sodium potassium tripolyphosphate or mixtures of sodium tripolyphosphate and potassium tripolyphosphate and sodium potassium tripolyphosphate may also be used in accordance with the invention.

Organic co builders, which may be used in the detergents and cleaning agents according to the invention, include, in particular, polycarboxylates or polycarboxylic acids, polymeric polycarboxylates, polyaspartic acid, polyacetals, optionally oxidized dextrins, other organic co builders (see below) and phosphonates. These classes of substances are described below.

Useful organic builders are, for example, the polycarboxylic acids usable in the form of their sodium salts, polycarboxylic acids in this context being understood to be carboxylic acids that carry more than one acid function. These include, for example, citric acid, adipic acid, succinic acid, glutaric acid, malic acid, tartaric acid, maleic acid, fumaric acid, sugar acids, aminocarboxylic acids, nitrilotriacetic acid (NTA), providing its use is not ecologically unsafe, and mixtures thereof. Preferred salts are the salts of polycarboxylic acids such as citric acid, adipic acid, succinic acid, glutaric acid, tartaric acid, sugar acids and mixtures thereof.

Acids per se can also be used. Besides their building effect, the acids also typically have the property of an acidifying component and, hence also serve to establish a relatively low and mild pH in washing or cleaning agents, when the pH, which results from the mixture of other components, is not wanted. Acids that are system-compatible and environmentally compatible such as citric acid, acetic acid, tartaric acid, malic acid, glycolic acid, succinic acid, glutaric acid, adipic acid, gluconic acid and mixtures thereof are particularly mentioned in this regard. However, mineral acids, particularly sulfuric acid or bases, particularly ammonium or alkali metal hydroxides can also serve as pH regulators. These types of regulators are preferably comprised in the inventive agents in amounts of not more than 20 wt. %, particularly from 1.2 wt. % to 17 wt. %.

Other suitable builders are polymeric polycarboxylates, i.e. for example the alkali metal salts of polyacrylic or polymethacrylic acid, for example those with a relative molecular weight of 500 to 70 000 g/mol.

The molecular weights mentioned in this specification for polymeric polycarboxylates are weight-average molecular weights Mw of the particular acid form which, fundamentally, were determined by gel permeation chromatography (GPC), equipped with a UV detector. The measurement was carried out against an external polyacrylic acid standard, which provides realistic molecular weight values by virtue of its structural similarity to the polymers investigated. These values differ significantly from the molecular weights measured against polystyrene sulfonic acids as the standard. The molecular weights measured against polystyrene sulfonic acids are generally significantly higher than the molecular weights mentioned in this specification.

Particularly suitable polymers are polyacrylates, which preferably have a molecular weight of 2000 to 20 000 g/mol. By virtue of their superior solubility, preferred representatives of this group are the short-chain polyacrylates, which have molecular weights of 2000 to 10 000 g/mol and, more particularly, 3000 to 5000 g/mol.

Further suitable copolymeric polycarboxylates are particularly those of acrylic acid with methacrylic acid and of acrylic acid or methacrylic acid with maleic acid. Copolymers of acrylic acid with maleic acid, which comprise 50 to 90 wt. % acrylic acid and 50 to 10 wt. % maleic acid, have proven to be particularly suitable. Their relative molecular weight, based on free acids, generally ranges from 2 000 to 70 000 g/mol, preferably 20 000 to 50 000 g/mol and especially 30 000 to 40 000 g/mol. The (co)polymeric polycarboxylates can be used either as powders or as aqueous solutions. The (co)polymeric polycarboxylate content of the compositions is preferably from 0.5 to 20% by weight, in particular from 1 to 10% by weight.

In order to improve the water solubility, the polymers can also comprise allylsulfonic acids, such as for example, allyloxybenzene sulfonic acid and methallyl sulfonic acid as monomers.

Other particularly preferred polymers are biodegradable polymers of more than two different monomer units, for example those which contain salts of acrylic acid and maleic acid and vinyl alcohol or vinyl alcohol derivatives as monomers or those which contain salts of acrylic acid and 2-alkylallyl sulfonic acid and sugar derivatives as monomers.

Other preferred copolymers are those, which preferably contain acrolein and acrylic acid/acrylic acid salts or acrolein and vinyl acetate as monomers.

Similarly, other preferred builders are polymeric amino dicarboxylic acids, salts or precursors thereof. Polyaspartic acids or their salts and derivatives are particularly preferred.

Further preferred builders are polyacetals that can be obtained by treating dialdehydes with polyol carboxylic acids that possess 5 to 7 carbon atoms and at least 3 hydroxyl groups. Preferred polyacetals are obtained from dialdehydes like glyoxal, glutaraldehyde, terephthalaldehyde as well as their mixtures and from polycarboxylic acids like gluconic acid and/or glucoheptonic acid.

Further suitable organic builders are dextrins, for example oligomers or polymers of carbohydrates that can be obtained by the partial hydrolysis of starches. The hydrolysis can be carried out using typical processes, for example acidic or enzymatic catalyzed processes. The hydrolysis products preferably have average molecular weights in the range 400 to 500 000 g/mol. A polysaccharide with a dextrose equivalent (DE) of 0.5 to 40 and, more particularly, 2 to 30 is preferred, the DE being an accepted measure of the reducing effect of a polysaccharide by comparison with dextrose, which has a DE of 100. Both maltodextrins with a DE between 3 and 20 and dry glucose syrups with a DE between 20 and 37 and also so-called yellow dextrins and white dextrins with relatively high molecular weights of 2000 to 30 000 g/mol may be used.

The oxidized derivatives of such dextrins concern their reaction products with oxidizing agents that are capable of oxidizing at least one alcohol function of the saccharide ring to the carboxylic acid function. Particularly preferred organic builders for inventive compositions are oxidized starches and their derivatives from the applications EP 472 042, WO 97/25399, and EP 755 944.

Oxydisuccinates and other derivatives of disuccinates, preferably ethylenediamine disuccinate are also further suitable cobuilders. Ethylenediamine-N,N′-disuccinate (EDDS) is preferably used here in the form of its sodium or magnesium salts. In this context, glycerine disuccinates and glycerine trisuccinates are also preferred. Suitable addition quantities in zeolite-containing and/or silicate-containing formulations range between 3 and 15% by weight.

Other useful organic co-builders are, for example, acetylated hydroxycarboxylic acids and salts thereof which may optionally be present in lactone form and which contain at least 4 carbon atoms, at least one hydroxyl group and at most two acid groups.

The phosphonates represent a further class of substances with cobuilder properties. In particular, they are hydroxyalkane phosphonates or aminoalkane phosphonates. Among the hydroxyalkane phosphonates, 1-hydroxyethane-1,1-diphosphonate (HEDP) is of particular importance as the cobuilder. It is normally added as the sodium salt, the disodium salt reacting neutral and the tetrasodium salt reacting alkaline (pH 9). Ethylenediamine tetramethylene phosphonate (EDTMP), diethylenetriamine pentamethylene phosphonate (DTPMP) and their higher homologs are preferably chosen as the aminoalkane phosphonates. They are preferably added in the form of the neutral-reacting sodium salts, e.g. as the hexasodium salt of EDTMP or as the hepta and octasodium salt of DTPMP. Of the class of phosphonates, HEDP is preferably used as the builder. The aminoalkane phosphonates additionally possess a pronounced ability to complex heavy metals. Accordingly, it can be preferred, particularly where the agents also contain bleach, to use aminoalkane phosphonates, particularly DTPMP, or mixtures of the mentioned phosphonates.

In addition, any compounds capable of forming complexes with alkaline earth metal ions may be used as co-builders.

Builders can be comprised in the inventive washing or cleaning agents optionally in quantities of up to 90% by weight. They are preferably comprised in quantities of up to 75% by weight. Inventive laundry detergents possess builder contents of particularly 5 wt. % to 50 wt. %. In inventive compositions for cleaning hard surfaces, in particular for automatic dishwashing of tableware, the content of builders is particularly 5 wt. % to 88 wt. %, wherein in this type of composition, no water-insoluble builders are employed. In a preferred embodiment, the inventive composition, particularly for automatic dishwashers, comprises 20 wt. % to 40 wt. % of water-soluble organic builders, particularly alkali citrate, 5 wt. % to 15 wt. % alkali carbonate and 20 wt. % to 40 wt. % alkali disilicate.

Solvents that can be added to the liquid to gel-like compositions of laundry detergent and cleaning compositions originate, for example, from the group of mono- or polyhydric alcohols, alkanolamines or glycol ethers, in so far that they are miscible with water in the defined concentrations. Preferably, the solvents are selected from ethanol, n- or i-propanol, butanols, ethylene glycol methyl ether, ethylene glycol ethyl ether, ethylene glycol propyl ether, ethylene glycol mono-n-butyl ether, diethylene glycol methyl ether, diethylene glycol ethyl ether, propylene glycol methyl-, -ethyl- or -propyl ether, dipropylene glycol methyl-, or -ethyl ether, methoxy-, ethoxy- or butoxy triglycol, 1-butoxyethoxy-2-propanol, 3-methyl-3-methoxybutanol, propylene glycol t-butyl ether as well as mixtures of these solvents.

Solvents can be employed in the inventive liquid to gel-like laundry detergents and cleaning compositions in amounts between 0.1 and 20 wt. %, preferably, however below 15 wt. % and particularly below 10 wt. %.

One or more thickeners or thickener systems can be added to the inventive composition to adjust the viscosity. These high molecular weight substances, which are also called swelling agents, soak up mostly liquids, thereby swelling up and subsequently transform into viscous, real or colloidal solutions.

Suitable thickeners are inorganic or polymeric organic compounds. The inorganic thickeners include, for example, polysilicic acids, mineral clays like montmorillonite, zeolites, silicic acids and bentonites. The organic thickeners come from the groups of natural polymers, derivatives of natural polymers and synthetic polymers. Exemplary, naturally occurring polymers that can be used as thickeners are agar, carrageen, tragacanth, gum Arabic, alginates, pectins, polyoses, guar meal, locust tree bean flour, starches, dextrins, gelatines and casein. Modified natural products that are used as thickeners are mainly derived from the group of the modified starches and celluloses. Examples can be cited as carboxymethyl cellulose and other cellulose ethers, hydroxyethyl- and hydroxypropyl cellulose as well as flour ether. Totally synthetic thickeners are polymers such as polyacrylics and polymethacrylics, vinyl polymers, polycarboxylic acids, polyethers, polyimines, polyamides and polyurethanes.

The thickeners can be comprised in amounts up to 5 wt. %, preferably from 0.05 to 2 wt. %, and particularly preferably from 0.1 to 1.5 wt. %, based on the finished preparation.

The washing or cleaning agents according to the invention can optionally comprise further typical ingredients—sequestering agents, electrolytes and further auxiliaries, such as optical brighteners, redeposition inhibitors, silver corrosion inhibitors, color transfer inhibitors, foam inhibitors, abrasives, dyes and/or fragrances, as well as antimicrobial agents, UV absorbers and/or enzyme stabilizers.

The detergents for textiles may contain derivatives of diaminostilbene disulfonic acid or alkali metal salts thereof as optical brighteners. Suitable optical brighteners are, for example, salts of 4,4′-bis-(2-anilino-4-morpholino-1,3,5-triazinyl-6-amino)stilbene-2,2′-disulfonic acid or compounds of similar structure which contain a diethanolamino group, a methylamino group, an anilino group or a 2-methoxyethylamino group instead of the morpholino group. Optical brighteners of the substituted diphenylstyryl type may also be present, for example the alkali metal salts of 4,4′-bis(2-sulfostyryl)diphenyl, 4,4′-bis(4-chloro-3-sulfostyryl)diphenyl or 4-(4-chlorostyryl)-4′-(2-sulfostyryl)diphenyl. Mixtures of the mentioned optical brighteners may also be used.

Graying inhibitors have the task of ensuring that the dirt removed from the textile fibers is held suspended in the wash liquid. Water-soluble colloids of mostly organic nature are suitable for this, for example starch, glue, gelatines, salts of ether carboxylic acids or ether sulfonic acids of starches or celluloses, or salts of acidic sulfuric acid esters of celluloses or starches. Water-soluble, acid group-containing polyamides are also suitable for this purpose. Moreover, aldehyde starches, for example, can be used instead of the abovementioned starch derivatives. Preference, however, is given to the use of cellulose ethers such as carboxymethyl cellulose (Na salt), methyl cellulose, hydroxyalkyl cellulose, and mixed ethers such as methyl hydroxyethyl cellulose, methyl hydroxypropyl cellulose, methyl carboxymethyl cellulose and mixtures thereof, which can be added, for example in amounts of 0.1 to 5 wt. %, based on the agent.

In order to realize a silver corrosion protection, silver protectors for tableware can be added to the inventive cleaning compositions. Benzotriazoles, ferric chloride or CoSO₄, for example, are known from the prior art. As is known from the European Patent EP 0 736 084 B1, for example, particularly suitable silver corrosion inhibitors for general use with enzymes are salts and/or complexes of manganese, titanium, zirconium, hafnium, vanadium, cobalt or cerium, in which the cited metals exist in the valence states II, III, IV, V or VI. Examples of these types of compounds are MnSO₄, V₂O₅, V₂O₄, VO₂, TiOSO₄, K₂TiF₆, K₂ZrF₆, Co(NO₃)₂, Co(NO₃)₃ and mixtures thereof.

Soil repellents are mostly polymers that when used in a laundry detergent, lend the fibers soil-repelling properties and/or support the soil repellent capabilities of the conventional ingredients. A comparable effect can also be observed when they are added in cleaning compositions for hard surfaces.

Particularly effective and well-known soil release agents are copolyesters with dicarboxylic acid, alkylene glycol and polyalkylene glycol units. Examples of these are copolymers or mixed polymers of polyethylene terephthalate and polyoxyethylene glycol (DT 16 17 141 and DT 22 00 911). German Offenlegungsschrift DT 22 53 063 cites acidic compositions, which inter alia comprise a copolymer of a dibasic acid and an alkylene or cycloalkylene polyglycol. Polymers of ethylene terephthalate and polyethylene oxide terephthalate and their use in laundry detergents are described in the German texts DE 28 57 292 and DE 33 24 258 and the European Patent EP 0 253 567. The European Patent EP 066 944 relates to compositions, which contain a copolyester of ethylene glycol, polyethylene glycol, aromatic dicarboxylic acids and sulfonated aromatic dicarboxylic acids in defined molar ratios. Polyesters, end-capped with methyl or ethyl groups, with ethylene and/or propylene terephthalate units and polyethylene oxide terephthalate units and laundry detergents that comprise such a soil-release polymer are known from EP 0 185 427. The European Patent EP 0 241 984 relates to a polyester, which in addition to oxyethylene groups and terephthalic acid units also comprises substituted ethylene units as well as glycerine units. Polyesters are known from EP 0 241 985 which comprise, beside oxyethylene groups and terephthalic acid units, 1,2-propylene, 1,2-butylene and/or 3-methoxy-1,2-propylene groups as well as glycerine units, and are end-capped with C₁ to C₄ alkyl groups. Polyesters with polypropylene terephthalate units and polyoxyethylene terephthalate units, at least partially end-capped with C₁₋₄ alkyl or acyl groups, are known from the European Patent application EP 0 272 033. The European Patent EP 0 274 907 describes soil-release polyesters containing terephthalate end-capped with sulfoethyl groups. According to the European Patent application EP 0 357 280, soil-release polyesters with terephthalate units, alkylene glycol units and poly-C₂₋₄ glycol units are manufactured by sulfonation of the unsaturated end groups. The international patent application WO 95/32232 relates to acidic, aromatic polyesters that are capable of releasing soil. For cotton materials, non-polymeric soil repellent active substances with a plurality of functional units are known from the international patent application WO 97/31085: A first unit, which can be cationic, for example, is able to be adsorbed onto the cotton surface by electrostatic attraction, and a second unit, which is designed to be hydrophobic, is responsible for the retention of the active agent at the water/cotton interface.

Color transfer inhibitors that can be used in inventive detergents for textiles particularly include polyvinyl pyrrolidones, polyvinyl imidazoles, polymeric N-oxides such as polyvinyl pyridine-N-oxide and copolymers of vinyl pyrrolidone with vinyl imidazole.

On using the agents in automatic cleaning processes, it can be advantageous to add foam inhibitors. Suitable foam inhibitors include for example, soaps of natural or synthetic origin, which have a high content of C₁₈-C₂₄ fatty acids.

Suitable non-surface-active types of foam inhibitors are, for example, organopolysiloxanes and mixtures thereof with microfine, optionally silanized silica and also paraffins, waxes, microcrystalline waxes and mixtures thereof with silanized silica or bis-stearyl ethylenediamide. Mixtures of various foam inhibitors, for example mixtures of silicones, paraffins or waxes, are also used with advantage. Preferably, the foam inhibitors, especially silicone-containing and/or paraffin-containing foam inhibitors, are loaded onto a granular, water-soluble or dispersible carrier material. Especially in this case, mixtures of paraffins and bis stearyl ethylenediamides are preferred.

An inventive cleaning composition for hard surfaces can moreover comprise abrasive ingredients, especially from the group comprising quartz meal, wood flour, plastic powder, chalk and microspheres as well as their mixtures. Abrasives are preferably comprised in the inventive cleaning compositions in amounts of not more than 20 wt. %, particularly from 5 wt. % to 15 wt. %.

Colorants and fragrances may be added to the laundry detergents and cleaning compositions in order to improve the esthetic impression created by the products and to provide the consumer not only with the required performance but also with a visually and sensorially “typical and unmistakable” product. Suitable perfume oils or fragrances include individual odoriferous compounds, for example synthetic products of the ester, ether, aldehyde, ketone, alcohol and hydrocarbon type. Odoriferous compounds of the ester type are, for example, benzyl acetate, phenoxyethyl isobutyrate, p-tert.-butylcyclohexyl acetate, linalyl acetate, dimethylbenzyl carbinyl acetate, phenylethyl acetate, linalyl benzoate, benzyl formate, ethylmethylphenyl glycinate, allylcyclohexyl propionate, styrallyl propionate and benzyl salicylate. The ethers include, for example, benzyl ethyl ether; the aldehydes include, for example, the linear alkanals containing 8 to 18 carbon atoms, citral, citronellal, citronellyloxyacetaldehyde, cyclamen aldehyde, hydroxycitronellal, lilial and bourgeonal; the ketones include, for example, the ionones, α-isomethyl ionone and methyl cedryl ketone; the alcohols include anethol, citronellol, eugenol, geraniol, linalool, phenylethyl alcohol and terpineol and the hydrocarbons include, above all, the terpenes, such as limonene and pinene. However, mixtures of various odoriferous substances, which together produce an attractive fragrant note, are preferably used. Perfume oils such as these may also contain natural odoriferous mixtures obtainable from vegetal sources, for example pine, citrus, jasmine, patchouli, rose or ylang-ylang oil. Also suitable are muscatel oil, oil of sage, chamomile oil, clove oil, melissa oil, mint oil, cinnamon leaf oil, lime blossom oil, juniper berry oil, vetivert oil, olibanum oil, galbanum oil and laudanum oil and orange blossom oil, neroli oil, orange peel oil and sandalwood oil. Normally the content of dyes lies below 0.01 wt. %, while fragrances can make up to 2 wt. % of the total formulation of the laundry detergent and cleaning compositions.

The fragrances may be directly incorporated in the laundry detergent or cleaning composition, although it can also be of advantage to apply the fragrances on carriers, which reinforce the adsorption of the perfume on the washing and thereby ensuring a long-lasting fragrance on the textiles by decreasing the release of the fragrance, especially for treated textiles. Suitable carrier materials are, for example, cyclodextrins, the cyclodextrin/perfume complexes optionally being coated with other auxiliaries. A further preferred carrier for fragrances is the described zeolite X, which instead of or in mixtures with surfactants can also take up fragrances. Accordingly, preferred laundry detergents and cleaning compositions comprise the described zeolite X and fragrances that are preferably at least partially absorbed on the zeolite.

Preferred colorants, which are not difficult for the expert to choose, have high storage stability, are not affected by the other ingredients of the composition or by light and do not have any pronounced substantivity for the textile fibers being treated, so as not to color them.

To control microorganisms, the laundry detergent or cleaning compositions may contain antimicrobial agents. Depending on the antimicrobial spectrum and the action mechanism, antimicrobial agents are classified as bacteriostatic agents and bactericides, fungistatic agents and fungicides, etc. Important substances from these groups are for example benzalkonium chlorides, alkylaryl sulfonates, halophenols and phenol mercury acetate. In the present context of the inventive teaching, the expressions, “antimicrobial activity” and “antimicrobial agent” have the usual technical meanings as defined, for example, by K. H. Wallhäuβer in “Praxis der Sterilisation, Desinfektion—Konservierung Keimidentifizierung—Betriebshygiene” (5th Edition, Stuttgart/New York: Thieme, 1995), any of the substances with antimicrobial activity described therein being usable. Suitable antimicrobial agents are preferably selected from the groups of alcohols, amines, aldehydes, antimicrobial acids and salts thereof, carboxylic acid esters, acid amides, phenols, phenol derivatives, diphenyls, diphenylalkanes, urea derivatives, oxygen and nitrogen acetals and formals, benzamidines, isothiazolines, phthalimide derivatives, pyridine derivatives, antimicrobial surface-active compounds, guanidines, antimicrobial amphoteric compounds, quinolines, 1,2-dibromo-2,4-dicyanobutane, iodo-2-propyl butyl carbamate, iodine, iodophores, peroxy compounds, halogen compounds and mixtures of the above.

The antimicrobials can be selected from: ethanol, n-propanol, i-propanol, 1,3-butane diol, phenoxyethanol, 1,2-propylene glycol, glycerin, undecylenic acid, benzoic acid, salicylic acid, dihydracetic acid, o-phenylphenol, N-methylmorpholine-acetonitrile (MMA), 2-benzyl-4-chlorophenol, 2,2′-methylenebis-(6-bromo-4-chlorophenol), 4,4′-dichloro-2′-hydroxydiphenyl ether (dichlosan), 2,4,4′-trichloro-2′-hydroxydiphenyl ether (trichlosan), chlorhexidine, N-(4-chlorophenyl)-N-(3,4-dichlorophenyl)-urea, N,N′-(1,10-decanediyldi-1-pyridinyl-4-ylidene)-bis-(1-octamine) dihydrochloride, N,N′-bis-(4-chlorophenyl)-3,12-diimino-2,4,11,13-tetraaza-tetradecanediimide amide, glucoprotamines, surface-active antimicrobial quaternary compounds, guanidines, including the bi- and polyguanidines, such as for example 1,6-bis(2-ethylhexylbiguanidohexane) dihydrochloride, 1,6-di-(N₁,N₁′-phenyldiguanido-N₅,N₅′)hexane tetrahydrochloride, 1,6-di-(N₁,N₁-phenyl-N₁,N₁-methyldiguanido-N₅,N₅)hexane dihydrochloride, 1,6-di-(N₁,N₁′-o-chlorophenyldiguanido-N₅,N₅′)hexane dihydrochloride, 1,6-di-(N₁,N′₁-2,6-dichlorophenyldiguanido-N₅,N₅′)hexane dihydrochloride, 1,6-di-[N₁,N₁′-β-(p-methoxyphenyl) diguanido-N₅,N₅′]hexane dihydrochloride, 1,6-di-(N₁,N₁′-α-methyl-β-phenyldiguanido-N₅,N₅′)hexane dihydrochloride, 1,6-di-(N₁,N₁′-p-nitrophenyldiguanido-N₅,N₅′)hexane dihydrochloride, ω:ω-di-(N₁,N₁′-phenyldiguanido-N₅,N₅′)di-n-propyl ether dihydrochloride, ω:ω-di-(N₁,N₁′-p-chlorophenyldiguanido-N₅,N₅′)di-n-propyl ether tetrahydrochloride, 1,6-di-(N₁,N₁′-2,4-dichlorophenyldiguanido-N₅,N₅′)hexane tetrahydrochloride, 1,6-di-(N₁,N₁′-p-methylphenyldiguanido-N₅,N₅′)hexane dihydrochloride, 1,6-di-(N₁,N₁′-2,4,5-trichlorophenyldiguanido-N₅,N₅′)hexane tetrahydrochloride, 1,6-di-[N₁,N₁′-α-(p-chlorophenyl)ethyldiguanido-N₅,N₅′]hexane dihydrochloride, ω:ω-di-(N₁,N₁′-p-chlorophenyldiguanido-N₅,N₅′)m-xylene dihydrochloride, 1,12-di-(N₁,N₁′-p-chlorophenyldiguanido-N₅,N₅′)dodecane dihydrochloride, 1,10-di-(N₁,N₁′-phenyldiguanido-N₅,N₅′)decane tetrahydrochloride, 1,12-di-(N₁,N₁′-phenyldiguanido-N₅,N₅′)dodecane tetrahydrochloride, 1,6-di-(N₁,N₁′-o-chlorophenyldiguanido-N₅,N₅′)hexane dihydrochloride, 1,6-di-(N₁,N₁-o-chlorophenyldiguanido-N₅,N₅′)hexane tetrahydrochloride, ethylene-bis-(1-tolylphenylbiguanide), ethylene-bis-(p-tolylphenylbiguanide), ethylene-bis-(3,5-dimethylphenylbiguanide), ethylene-bis-(p-tert-amylphenylbiguanide), ethylene-bis-(nonylphenylbiguanide), ethylene-bis-(phenylbiguanide), ethylene-bis-(N-butylphenylbiguanide), ethylene-bis-(2,5-diethoxyphenylbiguanide), ethylene-bis(2,4-dimethylphenylbiguanide), ethylene-bis-(o-diphenylbiguanide), ethylene-bis-(mixed amylnaphthylbiguanide), N-butylethylene-bis-(phenylbiguanide), trimethylene bis(o-tolylbiguanide), N-butyltrimethylene-bis-(phenylbiguanide) and the corresponding salts such as acetates, gluconates, hydrochlorides, hydrobromides, citrates, bisulfites, fluorides, polymaleates, N-coco alkyl sarcinosates, phosphites, hypophosphites, perfluorooctanoates, silicates, sorbates, salicylates, maleates, tartrates, fumarates, ethylenediaminetetraacetates, iminodiacetates, cinnamates, thiocyanates, arginates, pyromellitates, tetracarboxybutyrates, benzoates, glutarates, monofluorophosphates, perfluoropropionates as well as any mixtures thereof. Furthermore, halogenated xylene- and cresol derivatives are suitable, such as p-chloro-meta-cresol, p-chloro-meta-xylene, as well as natural antimicrobial active agents of plant origin (e.g. from spices or aromatics), animal as well as microbial origin. Furthermore, halogenated xylene- and cresol derivatives are suitable, such as p-chloro-meta-cresol, p-chloro-meta-xylene, as well as natural antimicrobial active agents of plant origin (e.g. from spices or aromatics), animal as well as microbial origin. Preferred antimicrobial agents are antimicrobial surface-active quaternary compounds, a natural antimicrobial agent of vegetal origin and/or a natural antimicrobial agent of animal origin and, most preferably, at least one natural antimicrobial agent of vegetal origin from the group comprising caffeine, theobromine and theophylline and essential oils, such as eugenol, thymol and geraniol, and/or at least one natural antimicrobial agent of animal origin from the group comprising enzymes, such as protein from milk, lysozyme and lactoperoxidase and/or at least one antimicrobial surface-active quaternary compound containing an ammonium, sulfonium, phosphonium, iodonium or arsonium group, peroxy compounds and chlorine compounds. Substances of microbial origin, so-called bacteriozines, may also be used.

The quaternary ammonium compounds (QUATS) suitable as antimicrobial agents have the general formula (R¹)(R²)(R³)(R⁴)N⁺X⁻, in which R¹ to R⁴ may be the same or different and represent C₁₋₂₂ alkyl groups, C₇₋₂₈ aralkyl groups or heterocyclic groups, two or—in the case of an aromatic compound, such as pyridine—even three groups together with the nitrogen atom forming the heterocycle, for example a pyridinium or imidazolinium compound, and X⁻ represents halide ions, sulfate ions, hydroxide ions or similar anions. In the interests of optimal antimicrobial activity, at least one of the substituents preferably has a chain length of 8 to 18 and, more preferably, 12 to 16 carbon atoms.

QUATS can be obtained by reacting tertiary amines with alkylating agents such as, for example, methyl chloride, benzyl chloride, dimethyl sulfate, dodecyl bromide and also ethylene oxide. The alkylation of tertiary amines having one long alkyl chain and two methyl groups is particularly easy. The quaternization of tertiary amines containing two long chains and one methyl group can also be carried out under mild conditions using methyl chloride. Amines containing three long alkyl chains or hydroxy-substituted alkyl chains lack reactivity and are preferably quaternized with dimethyl sulfate.

Suitable QUATS are, for example, Benzalkonium chloride (N-alkyl-N,N-dimethylbenzyl ammonium chloride, CAS No. 8001-54-5), Benzalkon B (m,p-dichlorobenzyl dimethyl-C12-alkyl ammonium chloride, CAS No. 58390-78-6), Benzoxonium chloride (benzyldodecyl-bis-(2-hydroxyethyl)ammonium chloride), Cetrimonium bromide (N-hexadecyl-N,N-trimethyl ammonium bromide, CAS No. 57-09-0), Benzetonium chloride (N,N-di-methyl-N-[2-[2-[p-(1,1,3,3-tetramethylbutyl)-phenoxy]ethoxy]ethyl]-benzylammonium chloride, CAS No. 121-54-0), dialkyl dimethyl ammonium chlorides, such as di-n-decyldimethyl ammonium chloride (CAS No. 7173-51-5-5), didecyldimethyl ammonium bromide (CAS No. 2390-68-3), dioctyl dimethylammonium chloride, 1-cetylpyridinium chloride (CAS No. 123-03-5) and thiazoline iodide (CAS No. 15764-48-1) and mixtures thereof. Particularly preferred QUATS are the Benzalkonium chlorides containing C₈₋₁₈ alkyl groups, more particularly C₁₂₋₁₄ alkylbenzyldimethylammonium chloride.

Benzalkonium halides and/or substituted Benzalkonium halides are commercially available, for example, as Barquat® from Lonza, Marquato® from Mason, Variquat® from Witco/Sherex and Hyamine® from Lonza and as Bardac® from Lonza. Other commercially obtainable antimicrobial agents are N-(3-chloroallyl)-hexaminium chloride, such as Dowicide® and Dowicil® from Dow, Benzethonium chloride, such as Hyamine® 1622 from Rohm & Haas, methyl Benzethonium chloride, such as Hyamine® 10× from Rohm & Haas, cetyl pyridinium chloride, such as Cepacolchloride from Merrell Labs.

The antimicrobial agents are used in quantities of 0.0001% by weight to 1% by weight, preferably 0.001% by weight to 0.8% by weight, particularly preferably 0.005% by weight to 0.3% by weight and most preferably 0.01 to 0.2% by weight.

The inventive laundry detergents or cleaning compositions may comprise UV absorbers that attach to the treated textiles and improve the light stability of the fibers and/or the light stability of the various ingredients of the formulation. UV-absorbers are understood to mean organic compounds, which are able to absorb UV radiation and emit the resulting energy in the form of longer wavelength radiation, for example as heat.

Compounds, which possess these desired properties, are for example, the efficient radiationless deactivating compounds and derivatives of benzophenone having substituents in position(s) 2- and/or 4. Also suitable are substituted benzotriazoles, acrylates, which are phenyl-substituted in position 3 (cinnamic acid derivatives optionally with cyano groups in position 2), salicylates, organic Ni complexes, as well as natural substances such as umbelliferone and the endogenous urocanic acid. The biphenyl and above all the stilbene derivatives such as for example those described in EP 0 728 749 A and commercially available as Tinosorb® FD or Tinosorb® FR from Ciba, are of particular importance. As UV-B absorbers can be cited: 3-benzylidenecamphor or 3-benzylidenenorcamphor and its derivatives, for example 3-(4-methylbenzylidene) camphor, as described in the EP 0693471 B1; 4-aminobenzoic acid derivatives, preferably 4-(dimethylamino)benzoic acid, 2-ethylhexyl ester, 4-(dimethylamino)benzoic acid, 2-octyl ester and 4-(dimethylamino)benzoic acid, amyl ester; esters of cinnamic acid, preferably 4-methoxycinnamic acid, 2-ethylhexyl ester, 4-methoxycinnamic acid, propyl ester, 4-methoxycinnamic acid, isoamyl ester, 2-cyano-3,3-phenylcinnamic acid, 2-ethylhexyl ester (octocrylene); esters of salicylic acid, preferably salicylic acid, 2-ethylhexyl ester, salicylic acid, 4-isopropylbenzyl ester, salicylic acid, homomethyl ester; derivatives of benzophenone, preferably 2-hydroxy-4-methoxybenzophenone, 2-hydroxy-4-methoxy-4′-methylbenzophenone, 2,2′-dihydroxy-4-methoxybenzophenone; esters of benzalmalonic acid, preferably 4-methoxybenzmalonic acid, di-2-ethylhexylester; triazine derivatives, such as, for example 2,4,6-trianilino-(p-carbo-2′-ethyl-1′-hexyloxy)-1,3,5-triazine and octyl triazone, as described in EP 0818450 A1 or dioctyl butamidotriazone (Uvasorb® HEB); propane-1,3-dione, such as for example 1-(4-tert.-butylphenyl)-3-(4′-methoxyphenyl)propane-1,3-dione; ketotricyclo(5.2.1.0) decane derivatives, as described in EP 0694521 B1. Further suitable are 2-phenylbenzimidazole-5-sulfonic acid and its alkali, alkaline earth, ammonium, alkylammonium, alkanolammonium and glucammonium salts; sulfonic acid derivatives of benzophenones, preferably 2-hydroxy-4-methoxybenzophenone-5-sulfonic acid and its salts; sulfonic acid derivatives of 3-benzylidenecamphor, as for example 4-(2-oxo-3-bornylidenemethyl)benzene sulfonic acid and 2-methyl-5-(2-oxo-3-bornylidene) sulfonic acid and their salts.

Typical UV-A filters particularly include derivatives of benzoylmethane, such as, for example 1-(4′-tert.-butylphenyl)-3-(4′-methoxyphenyl)propane-1,3-dione, 4-tert.-butyl-4′-methoxydibenzoylmethane (Parsol 1789), 1-phenyl-3-(4′-isopropylphenyl)-propane-1,3-dione as well as enamine compounds, as described in the DE 1 971 2033 A1 (BASF). Naturally, the UV-A and UV-B filters can also be added as mixtures. Beside the cited soluble materials, insoluble, light protective pigments, namely finely dispersed, preferably, nano metal oxides or salts can also be considered for this task. Exemplary suitable metal oxides are particularly zinc oxide and titanium oxide and also oxides of iron, zirconium, silicon, manganese, aluminum and cerium as well as their mixtures. Silicates (talc), barium sulfate or zinc stearate can be added as salts. The oxides and salts are already used in the form of pigments for skin care and skin protecting emulsions and decorative cosmetics. Here, the particles should have a mean diameter of less than 100 nm, preferably between 5 and 50 nm and especially between 15 and 30 nm. They can be spherical, however elliptical or other non-spherical shaped particles can also be used. The pigments can also be surface treated, i.e. hydrophilized or hydrophobized. Typical examples are coated titanium dioxides, such as, for example Titandioxid Z 805 (Degussa) or Eusolex® T2000 (Merck); preferably, silicones and particularly preferably trialkoxy octylsilanes or Simethicones are used as the hydrophobic coating agents Preferably, micronized zinc oxide is used. Further suitable UV light protection filters may be found in the review by P. Finkel in SöFW-Journal 122 (1996) p. 543.

The UV absorbers are normally used in amounts of 0.01 wt. % to 5 wt. %, preferably from 0.03 wt. % to 1 wt. %.

To increase their washing or cleaning power, agents according to the invention can comprise, in addition to the inventive proteases, further enzymes, wherein in principle, any enzyme established for these purposes in the prior art may be used. These particularly include further proteases, amylases, lipases, hemicellulases, cellulases or oxidoreductases as well as preferably their mixtures. In principle, these enzymes are of natural origin; improved variants based on the natural molecules are available for use in laundry detergents and cleaning agents and accordingly they are preferred. The agents according to the invention preferably comprise enzymes in total quantities of 1×10⁻⁶ to 5 weight percent based on active protein.

Preferred additional proteases are those of the subtilisin type. Examples of these are subtilisins BPN′ and Carlsberg, the protease PB92, the subtilisins 147 and 309, the alkaline protease from Bacillus lentus, subtilisin DY and those enzymes of the subtilases no longer however classified in the stricter sense as subtilisins thermitase, proteinase K and the proteases TW3 und TW7. Subtilisin Carlsberg in further developed form is available under the trade name Alcalase® from Novozymes A/S, Bagsværd, Denmark. Subtilisins 147 and 309 are commercialized under the trade names Esperase® and Savinase® by the Novozymes company. Variants derived from the protease from Bacillus lentus DSM 5483 (WO 91/02792 A1) called BLAP® are described especially in WO 92/21760 A1, WO 95/23221 A1, WO 02/088340 A2 and WO 03/038082 A2. Further useable proteases from various Bacillus sp. and B. gibsonii strains emerge from the patent applications WO 03/054185, WO 03/056017, WO 03/055974 and WO 03/054184.

Further useable proteases are, for example, those enzymes available under the trade names Durazym®, Relase®, Everlase®, Nafizym, Natalase®, Kannase® and Ovozymes® from the Novozymes Company, those under the trade names Purafect®, Purafect® OxP and Properase® from Genencor, that under the trade name Protosol® from Advanced Biochemicals Ltd., Thane, India, that under the trade name Wuxi® from Wuxi Snyder Bioproducts Ltd., China, those under the trade names Proleather® and Protease P® from Amano Pharmaceuticals Ltd., Nagoya, Japan, and that under the designation Proteinase K-16 from Kao Corp., Tokyo, Japan.

Examples of useable amylases according to the invention are the α-amylases from Bacillus licheniformis, from B. amyloliquefaciens and from B. stearothermophilus, as well as their improved further developments for use in laundry detergents and cleaning compositions. The enzyme from B. licheniformis is available from the Novozymes Company under the name Termamyl® and from the Genencor Company under the name Purastar® ST. Further development products of this α-amylase are available from the Novozymes Company under the trade names Duramyl® and Termamyl® ultra, from the Genencor Company under the name Purastar® OxAm and from Daiwa Seiko Inc., Tokyo, Japan as Keistase®. The α-amylase from B. amyloliquefaciens is commercialised by the Novozymes Company under the name BAN®, and derived variants from the α-amylase from B. stearothermophilus under the names BSG® and Novamyl® also from the Novozymes Company. Additional commercial products that can be used are for example the Amylase-LT® and Stainzyme®, the latter also from the Novozymes company.

Moreover, for these purposes, attention should be drawn to the α-amylase from Bacillus sp. A 7-7 (DSM 12368) disclosed in the application WO 02/10356 A2 and the cyclodextrin-glucanotransferase (CGTase) from B. agaradherens (DSM 9948) described in the application WO 02/44350 A2.

Furthermore, the amylolytic enzymes are useable, which belong to the sequence space of α-amylase, described in the application WO 03/002711 A2 and those described in the application WO 03/054177 A2. Similarly, fusion products of the cited molecules are applicable, for example those from the application DE 10138753 A1.

Moreover, further developments of α-amylase from Aspergillus niger und A. oryzae available from the Company Novozymes under the trade name Fungamyl® are suitable. A further commercial product is the amylase-LT® for example.

The agents according to the invention can comprise lipases or cutinases, particularly due to their triglyceride cleaving activities, but also in order to produce in situ peracids from suitable preliminary steps. These include for example the available or further developed lipases originating from Humicola lanuginosa (Thermomyces lanuginosus), particularly those with the amino acid substitution D96L. They are commercialized, for example by the Novozymes Company under the trade names Lipolase®, Lipolase® Ultra, LipoPrime®, Lipozyme® and Lipex®. Moreover, suitable cutinases, for example are those that were originally isolated from Fusarium solani pisi and Humicola insolens. Likewise useable lipases are available from the Amano Company under the designations Lipase CE®, Lipase P®, Lipase B®, and Lipase CES®, Lipase AKG®, Bacillis sp. Lipase®, Lipase AP®, Lipase M-AP® and Lipase AML®. Suitable lipases or cutinases whose starting enzymes were originally isolated from Pseudomonas mendocina and Fusarium solanii are for example available from the Genencor Company. Further important commercial products that may be mentioned are the commercial preparations M1 Lipase® and Lipomax® originally from Gist-Brocades Company, and the commercial enzymes from the Meito Sangyo KK Company, Japan under the names Lipase MY-30®, Lipase OF® and Lipase PL® as well as the product Lumafast® from the Genencor Company.

Compositions according to the invention, particularly when they are destined for treating textiles, can comprise cellulases, according to their purpose, as pure enzymes, as enzyme preparations, or in the form of mixtures in which the individual components advantageously complement their various performances. Among these aspects of performance are particular contributions to primary washing performance, to secondary washing performance of the product, (anti-redeposition activity or inhibition of graying) and softening or brightening (effect on the textile), through to practicing a “stone washed” effect.

A usable, fungal endoglucanases (EG)-rich cellulase preparation, or its further developments are offered by the Novozymes Company under the trade name Celluzyme®. The products Endolase® and Carezyme® based on the 50 kD-EG, respectively 43 kD-EG from H. insolens DSM 1800 are also obtainable from Novozymes Company. The latter is based on the application WO 96/29397 A1. Performance enhanced cellulase variants emerge from the application WO 98/12307 A1, for example. It is equally possible to use the cellulases disclosed in the application WO 97/14804 A1; for example the 20 kD EG disclosed therein from Melanocarpus, and which is available under the trade names Ecostone® and Biotouch® from AB Enzymes, Finland. Further commercial products from the AB Enzymes Company are Econase® and Ecopulp®. Further suitable cellulases from Bacillus sp. CBS 670.93 and CBS 669.93 are disclosed in WO 96/34092 A2, the CBS 670.93 from Bacillus sp. being obtainable under the trade name Puradax® from the Genencor Company. Other commercial products from the Genencor Company are “Genencor detergent cellulase L” and IndiAge® Neutra.

The compositions according to the invention can comprise, besides the inventive polypeptides, additional enzymes especially for removing specific problem stains and which are summarized under the term hemicellulases. These include, for example mannanases, xanthanlyases, pectinlyases (=pectinases), pectinesterases, pectatlyases, xyloglucanases (=xylanases), pullulanases und β-glucanases. Suitable mannanases, for example are available under the names Gamanase® and Pektinex AR® from Novozymes Company, under the names Rohapec® B1 from AB Enzymes and under the names Pyrolase® from Diversa Corp., San Diego, Calif., USA. A suitable β-Glucanase from a B. alcalophilus emerges from the application WO 99/06573 A1, for example. β-Glucanase extracted from B. subtilis is available under the name Cereflo® from Novozymes Company.

To increase the bleaching action, the laundry detergents and cleaning agents according to the invention can comprise oxidoreductases, for example oxidases, oxygenases, katalases, peroxidases, such as halo-, chloro-, bromo-, lignin-, glucose- or manganese-peroxidases, dioxygenases or laccases (phenoloxidases, polyphenoloxidases). Suitable commercial products are Denilite® 1 and 2 from the Novozymes Company. Advantageously, additional, preferably organic, particularly preferably aromatic compounds are added that interact with the enzymes to enhance the activity of the oxidoreductases in question or to facilitate the electron flow (mediators) between the oxidizing enzymes and the stains over strongly different redox potentials.

The enzymes used in the agents according to the invention either stem originally from microorganisms, such as the species Bacillus, Streptomyces, Humicola, or Pseudomonas, and/or are produced according to known biotechnological processes using suitable microorganisms, such as by transgenic expression hosts of the species Bacillus or filamentary fungi.

Purification of the relevant enzymes follows conveniently using established processes such as precipitation, sedimentation, concentration, filtration of the liquid phases, microfiltration, ultrafiltration, mixing with chemicals, deodorization or suitable combinations of these steps.

The inventive polypeptides as well as the optionally additionally employed enzymes can be added to the inventive compositions in each established form known from the prior art. Included here, for example, are solid preparations obtained by granulation, extrusion or lyophilization, or particularly for liquid compositions or compositions in the form of gels, enzyme solutions, advantageously highly concentrated, of low moisture content and/or mixed with stabilizers.

As an alternative application form, these proteins can also be encapsulated, for example by spray drying or extrusion of the enzyme solution together with a preferably natural polymer or in the form of capsules, for example those in which the enzyme is embedded in a solidified gel, or in those of the core-shell type, in which an enzyme-containing core is coated with a water-, air- and/or chemical-impervious protective layer. Further active principles, for example stabilizers, emulsifiers, pigments, bleaches or colorants, can be applied in additional layers. Such capsules are made using known methods, for example by vibratory granulation or roll compaction or by fluidized bed processes. Advantageously, these types of granulates, for example with an applied polymeric film former are dust-free and as a result of the coating are storage stable.

In addition, it is possible to formulate two or more enzymes together, for example an inventive polypeptide and an additional enzyme, such that a single granulate exhibits a plurality of enzymatic activities.

A protein, especially also the inventive protein, comprised in an inventive composition can be protected, particularly in storage, against deterioration such as, for example inactivation, denaturation or decomposition, for example through physical influences, oxidation or proteolytic cleavage. An inhibition of the proteolysis is particularly preferred during microbial preparation of proteins and/or enzymes, particularly when the compositions also contain proteases. Preferred compositions according to the invention comprise stabilizers for this purpose.

One group of stabilizers is the reversible protease inhibitors. For this, benzamidine hydrochloride, borax, boric acids, boronic acids or their salts or esters are frequently used, above all derivatives with aromatic groups, for example ortho, meta or para substituted phenyl boronic acids, particularly 4-formylphenyl boronic acid or the salts or esters of the cited compounds. Peptide aldehydes, i.e. oligopeptides with a reduced C-terminus, particularly those from 2 to 50 monomers, are also used for this purpose. Ovomucoid and leupeptin, among others, belong to the peptidic reversible protease inhibitors. Specific, reversible peptide inhibitors for the protease subtilisin and fusion proteins from proteases and specific peptide inhibitors are also suitable.

Further enzyme stabilizers are amino alcohols such as mono-, di-, triethanol- and -propanolamine and their mixtures, aliphatic carboxylic acids up to C₁₂, such as, for example succinic acid, other dicarboxylic acids or salts of the cited acids. End-capped fatty acid amide alkoxylates are also suitable for this purpose. Certain organic acids used as builders can, as disclosed in WO 97/18287 additionally stabilize an included enzyme.

Lower aliphatic alcohols, but above all polyols such as, for example glycerine, ethylene glycol, propylene glycol or sorbitol, are other frequently used enzyme stabilizers. Di-glycerol phosphate also protects against denaturation by physical influences. Similarly, calcium and/or magnesium salts are used, such as, for example calcium acetate or calcium formate.

Polyamide oligomers or polymeric compounds such as lignin, water-soluble vinyl copolymers or cellulose ethers, acrylic polymers and/or polyamides stabilize the enzyme preparations inter alia against physical influences or pH variations. Polymers containing polyamine-N-oxide act simultaneously as enzyme stabilizers and as color transfer inhibitors. Other polymeric stabilizers are linear C₈-C₁₈ polyoxyalkylenes. Alkyl polyglycosides can also stabilize the enzymatic components of the inventive composition and are additionally capable of advantageously increasing their performance. Crosslinked nitrogen-containing compounds preferably perform a dual function as soil release agents and as enzyme stabilizers. A hydrophobic, non-ionic polymer stabilizes in particular an optionally present cellulase.

Reducing agents and antioxidants increase the stability of enzymes against oxidative decomposition; sulfur-containing reducing agents are commonly used here. Other examples are sodium sulfite and reducing sugars.

The use of combinations of stabilizers is particularly preferred, for example of polyols, boric acid and/or borax, the combination of boric acid or borate, reducing salts and succinic acid or other dicarboxylic acids or the combination of boric acid or borate with polyols or polyamino compounds and with reducing salts. The effect of peptide-aldehyde stabilizers is conveniently increased by the combination with boric acid and/or boric acid derivatives and polyols and even more by the additional effect of divalent cations, such as for example calcium ions.

Since agents of the invention can be provided in any conceivable form, polypeptides according to the invention in any formulations that are appropriate for addition to the particular agents, represent respective embodiments of the present invention. Examples thereof include liquid formulations, solid granules or capsules.

The encapsulated form is a way of protecting the enzymes or other ingredients against other components such as, for example, bleaching agents, or of making possible a controlled release. Depending on their size, said capsules are divided into milli-, micro- and nanocapsules, microcapsules being particularly preferred for enzymes. Such capsules are disclosed, for example, in the Patent applications WO 97/24177 and DE 199 18 267. A possible encapsulation method is to encapsulate the proteins, starting from a mixture of the protein solution with a solution or suspension of starch or a starch derivative, in this substance. Such an encapsulation process is described in the application WO 01/38471.

In the case of solid compositions, the proteins—inventive polypeptides just as the optionally comprised additional enzymes—may be used, for example, in dried, granulated and/or encapsulated form. They can be added separately, i.e. as one phase, or together with other ingredients in the same phase, with or without compaction. If microencapsulated, solid enzymes are used, then the water can be removed from the aqueous solutions resulting from the process by means of processes known from the prior art, such as spray-drying, centrifugation or by trans-dissolution. The particles obtained in this manner normally have a particle size between 50 and 200 μm.

Starting from protein recovery carried out according to the prior art, and preparation in a concentrated aqueous or non-aqueous solution, suspension or emulsion, but also in gel form or encapsulated or as a dried powder, the proteins can be added to liquid, gelled or pasty compositions of the invention. Such laundry detergents or cleaning compositions of the invention are usually prepared by simply mixing the ingredients, which may be introduced as solids or as solution into an automated mixer.

An inventive cleaning composition, in particular an inventive cleaner for hard surfaces, can also comprise one or more propellants, usually in an amount of 1 to 80 wt. %, preferably 1.5 to 30 wt. %, particularly 2 to 10 wt. %, particularly preferably 2.5 to 8 wt. %, above all 3 to 6 wt. %.

Propellants, according to the invention, are usually propellant gases, particularly liquefied or compressed gases. The choice depends on the product to be sprayed and the field of application. When using compressed gases such as nitrogen, carbon dioxide or nitrous oxide, which are generally insoluble in the liquid cleaning composition, the operating pressure is reduced each time the valve is actuated. Liquefied gases that are soluble in, or that themselves act as solvents for the cleaning composition, offer as propellants the advantage of a constant operating pressure and uniform dispersion, because the propellant evaporates in air and thereby expands several hundred times in volume.

Accordingly, the following are suitable propellants (names according to INCI): Butane, Carbon Dioxide, Dimethyl Carbonate, Dimethyl Ether, Ethane, Hydrochlorofluorocarbon 22, Hydrochlorofluorocarbon 142b, Hydrofluorocarbon 152a, Hydrofluorocarbon 134a, Hydrofluorocarbon 227ea, Isobutane, Isopentane, Nitrogen, Nitrous Oxide, Pentane, Propane. However, the use of chlorofluorocarbons (CFC) as propellants is preferably widely avoided and especially totally avoided due to their harmful effect on the ozone layer of the atmosphere that protects against harmful UV radiation.

Preferred propellants are liquefied gases. Liquid gases are gases that can be transformed from the gaseous into the liquid state at mostly already low pressures and 20° C. However liquid gases are particularly understood to be the hydrocarbons propane, propene, butane, butene, isobutane (2-methylpropane), isobutene (2-methylpropene, isobutylene) and their mixtures, which occur as by products from distilling and cracking oil in oil refineries as well as in natural gas processing in gasoline separation.

The cleaning composition particularly preferably comprises one or a plurality of propellants selected from propane, butane and/or isobutane, especially propane and butane, most preferably propane, butane and isobutane.

An important object of the enzyme preparation and particularly of the inventive polypeptide is, as listed above, the primary laundry performance. Apart from the primary washing performance, the proteases comprised in laundry detergents may further fulfil the function of activating, or, after an appropriate contact time, inactivating other enzymatic constituents by proteolytic cleavage. An embodiment of the present invention likewise relates to those agents containing capsules of protease-sensitive material, which capsules are hydrolyzed, for example, by proteins of the invention at the intended time and release their contents. Polypeptides of the invention can thus also be used for inactivation reactions, activation reactions or release reactions, in particular in multi phase agents.

The use of an inventive polypeptide for the activation, deactivation or release of ingredients of washing or cleaning agents is a further embodiment of this subject matter of the invention.

In a preferred embodiment, the composition containing an inventive polypeptide is designed in such a way that it can be used regularly as a conditioner, for example by adding it to the washing process, using it after washing or applying it independently of the washing. The desired effect is to obtain a smooth surface structure of the textile over a long period and/or to prevent and/or reduce damage to the fabric.

Processes, in which an inventive polypeptide is used in at least one of the process steps for the automatic cleaning of textiles or hard surfaces, constitute an independent subject of the invention.

In preferred processes for cleaning textiles or hard surfaces, the inventive polypeptide is employed in a quantity of 40 μg to 4 g, preferably from 50 μg to 3 g, particularly preferably from 100 μg to 2 g and quite particularly preferably from 200 μg to 1 g per application. All whole numbered and non-whole numbered values between these numbers are included.

These processes include both manual as well as automatic processes, automatic processes being preferred due to their more precise controllability that concerns for example the added quantities and contact times.

Processes for the cleaning of textiles are generally characterized in that various cleaning-active substances are applied to the material to be cleaned in a plurality of process steps and, after the contact time, are washed away, or that the material to be cleaned is treated in any other way with a laundry detergent or a solution of this detergent. The same applies to methods for cleaning any materials other than textiles, which are classified by the term hard surfaces. It is possible to add inventive proteins to at least one of the process steps of all conceivable washing or cleaning processes; accordingly, these processes then become embodiments of the present invention.

As preferred inventive polypeptides already naturally possess a protein-dissolving activity and also develop this in media that do not have any cleaning power, such as for example in a buffer, an individual partial step of such a process for automatic cleaning of textiles can consist of applying, if desired in addition to stabilizing compounds, salts or buffer substances, an inventive polypeptide as the single active component. This is a particularly preferred embodiment of the present invention.

In a further preferred embodiment of such processes, the inventive polypeptides in question are supplied in the context of one of the above listed formulations for inventive agents, preferably inventive washing or cleaning agents.

The use of one of the above described, inventive alkaline proteases for the cleaning of textiles or of hard surfaces is a separate subject matter of the invention.

Preferably, the above listed concentration ranges correspondingly apply to these uses.

Inventive proteases, particularly corresponding to the above-described characteristics and the above-described processes, can be used to remove protein-containing contaminants from textiles or from hard surfaces. Washing by hand or the manual removal of blemishes from textiles or from hard surfaces or the use in connection with an automatic process are exemplary embodiments.

In a preferred embodiment of this use, the inventive alkaline proteases in question are supplied in the context of one of the above listed formulations for inventive agents, preferably washing or cleaning agents.

Another subject matter of the present invention is also a product comprising an inventive composition or an inventive laundry detergent or cleaning composition, in particular an inventive cleaner for hard surfaces, and a spray dispenser. In this regard, the product can be either a single chamber container as well as a multi-chamber container, in particular a two-chamber container. The preferred spray dispenser is a manually operated spray dispenser, selected in particular from the group including aerosol spray dispensers (pressurized gas containers; also known inter alia as spray cans), self generated pressure spray dispensers, pump spray dispensers and trigger spray dispensers, particularly pump spray dispensers and trigger spray dispensers with a container made of transparent polyethylene or polyethylene terephthalate. Spray dispensers are extensively described in WO 96/04940 (Proctor & Gamble) and in the US patents cited therein concerning spray dispensers, all of which are referred to in this respect and their content is hereby incorporated in this application. Trigger spray dispensers and pump spray dispensers are advantageous in comparison with pressurized gas containers as no propellant need be employed. By means of attachments suitable for particles, (“nozzle-valves”) on the spray dispenser, the enzyme in this embodiment can also be optionally added in the form of immobilized particles to the composition and can thus be dosed as the cleaning foam.

While particular embodiments of the present invention have been illustrated and described, it would be obvious to those skilled in the art that various other changes and modifications can be made without departing from the spirit and scope of the invention.

Other than where otherwise indicated, or where required to distinguish over the prior art, all numbers expressing quantities of ingredients herein are to be understood as modified in all instances by the term “about”. As used herein, the words “may” and “may be” are to be interpreted in an open-ended, non-restrictive manner. At minimum, “may” and “may be” are to be interpreted as definitively including, but not limited to, the composition, structure, or act recited.

As used herein, and in particular as used herein to define the elements of the claims that follow, the articles “a” and “an” are synonymous and used interchangeably with “at least one” or “one or more,” disclosing or encompassing both the singular and the plural, unless specifically defined herein otherwise. The conjunction “or” is used herein in both in the conjunctive and disjunctive sense, such that phrases or terms conjoined by “or” disclose or encompass each phrase or term alone as well as any combination so conjoined, unless specifically defined herein otherwise.

The description of a group or class of materials as suitable or preferred for a given purpose in connection with the invention implies that mixtures of any two or more of the members of the group or class are equally suitable or preferred. Description of constituents in chemical terms refers unless otherwise indicated, to the constituents at the time of addition to any combination specified in the description, and does not necessarily preclude chemical interactions among the constituents of a mixture once mixed. Steps in any method disclosed or claimed need not be performed in the order recited, except as otherwise specifically disclosed or claimed.

Changes in form and substitution of equivalents are contemplated as circumstances may suggest or render expedient. Although specific terms have been employed herein, such terms are intended in a descriptive sense and not for purposes of limitation.

The following Examples further illustrate the preferred embodiments within the scope of the present invention, but are not intended to be limiting thereof. It is understood that the examples and embodiments described herein are for illustrative purposes only and that various modifications or changes in light thereof will be suggested to one skilled in the art without departing from the scope of the present invention. The appended claims therefore are intended to cover all such changes and modifications that are within the scope of this invention.

### EXAMPLES

All molecular-biological work was carried out by standard methods as can be found, for example, in the manual by Fritsch, Sambrook and Maniatis “Molecular cloning: a laboratory manual”, Cold Spring Harbour Laboratory Press, New York, 1989, or comparable specialist literature. Enzymes and kits were used according to the directions of the relevant manufacturer.

### Example 1

### Isolation and Identification of a Proteolytically Active Bacteria Strain

0.1 g of a core sample was suspended in 1 ml sterile NaCl and plated onto mill powder-containing agar plates (1.5% agar, 0.1% K₂HPO₄, 0.5% yeast extract, 1% peptone, 1% milk powder, 0.02% MgSO₄.7H₂O, 0.4% Na₂CO₃, pH 10) and incubated at 30° C. Using a zone of clearing a proteolytically active bacterium was isolated that was identified as Bacillus gibsonii by the Deutschen Sammlung von Mikroorganismen und Zellkulturen (DSMZ).

TABLE 1
Microbiological properties of the Bacillus gibsonii strain
(Determination by DSMZ)

| Property | Result |
|---|---|
| Cell form | rods |
| width [μm] | 0.9-1.1 |
| length [μm] | 2.0->3.0 |
| Spores | positive, oval |
| Swollen sporangium | negative |
| Anaerobic growth | negative |
| VP Reaction | negative |
| pH in VP Medium | 6.3 |
| Maximum Temperature | |
| Positive growth at ° C. | 30 |
| Negative growth at ° C. | 40 |
| Growth in | |
| medium pH 5.7 | negative |
| NaCI 2% | positive |
| 5% | positive |
| 7% | positive |
| 10%. | positive |
| Acid from (ASS) | |
| D-Glucose | weak |
| L-Arabinose | negative |
| D-Xylose | negative |
| D-Mannitol | weak |
| D-Fructose | weak |
| Gas from glucose | negative |
| Lecithinase | negative |
| Hydrolysis of | |
| Starch | negative |
| Gelatine | positive |
| Casein | negative |
| Tween 80 | negative |
| Tween 20 | negative |
| Tween 40 | negative |
| Tween 60 | negative |
| Esculin | |
| Esculin | |
| Utilization of | |
| Citrate (Koser) | Negative |
| Propionate | No growth |
| NO₂ from NO₃ | negative |
| Indole reaction | no growth |
| Phenylalaninedesaminase | negative |
| Argininedihydrolase | negative |
| Sample of the cellular fatty acids | Typical for genus Bacillus |
| Partial sequencing of the S-rDNA | 99.6% similarity with B. gibsonii |

### Example 2

### Cloning and Sequencing of the Mature Protease

The proteolytically active bacterium was cultivated in TBY-medium (0.5% NaCl, 0.5% yeast extract, 1% Trypton, pH 7.4) for 16 hours at 30° C. The total DNA of this bacterium was isolated, digested with the restriction enzyme Sau 3A and the obtained fragments cloned into the vector pAWA22. This is an expression vector derived from pBC16 for use in Bacillus species (Bernhard et al. (1978), J. Bacteriol., volume 133 (2), pp. 897-903). This vector was transformed into the host cell Bacillus subtilis DB 104 (Kawamura and Doi (1984), J. Bacteriol., volume 160 (1), pp. 442-444)

The transformants were initially regenerated on DM3 medium (8 g/l agar, 0.5 M succinic acid, 3.5 g/l K₂HPO₄, 1.5 g/l KH₂PO₄, 20 mM MgCl₂, 5 g/l casiamino acids, 5 g/l yeast extract, 6 g/l glucose, 0.1 g/l BSA) and then transferred to TBY skim milk plates (10 g/l peptone, 10 g/1 milk powder (see above), 5 g/l yeast, 5 g/l NaCl, 15 g/l agar). Clones with proteolytic activity were identified from their zones of lysis. One of the resulting clones with proteolytic activity was selected, and its plasmid was isolated and the insert was sequenced by standard methods.

The insert, approx. 1.5 kb in size, contained an open reading frame of about 1.2 kb. The sequence thereof is indicated in the sequence listing under the heading SEQ ID No. 1. It comprises 1152 bp. The amino acid sequence derived there from comprises 383 amino acids, followed by a stop codon. It is indicated in the sequence listing under SEQ ID No. 2. The first 114 amino acids thereof are probably not present in the mature protein, so that the envisaged length of the mature protein is 269 amino acids.

These sequences were compared with the protease sequences obtainable from generally accessible databases Swiss-Prot (Geneva Bioinformatics (GeneBio) S.A., Geneva, Switzerland; http://www.genebio.com/sprot.html) and GenBank (National Center for Biotechnology Information NCBI, National Institutes of Health, Bethesda, Md., USA). The most similar enzymes identified were the three summarized in Table 2 below.

TABLE 2
Homology of the alkaline protease from Bacillus gibsonii to the
most similar proteins.

| | | | Ident. | Ident. | | Ident. |
|---|---|---|---|---|---|---|
| | | | k. | m. | Ident. | m. |
| Enzyme | Organism | Source | DNA | DNA | Proprä. | Prot. |
| Subtilisin | Bacillus | DE102006022216 | 88 | 87 | 96 | 97 |
| HP302 | gibsonii | | | | | |
| Subtilisin | Bacillus | WO03/054184 | 88 | 86 | 95 | 96 |
| TI-1 | gibsonii | | | | | |
| Subtilisin | Bacillus | WO03/054185 | 88 | 84 | 92 | 91 |
| TII-5 | gibsonii | | | | | |

The meanings therein are:

| | |
|---|---|
| Source | document, in which the sequence is disclosed; |
| Ident. k. DNA | identity at the DNA-level for the complete DNA in %; |
| Ident. m. DNA | identity at the DNA-level for the DNA coding for the |
| | mature protein in %; |
| Ident. Proprä. | identity at the amino acid level, with respect to the |
| | propreprotein, in %; |
| Ident. m. Prot. | identity at the amino acid level, with respect to the |
| | mature protein, in %; |
| n. | not listed in the databanks. |

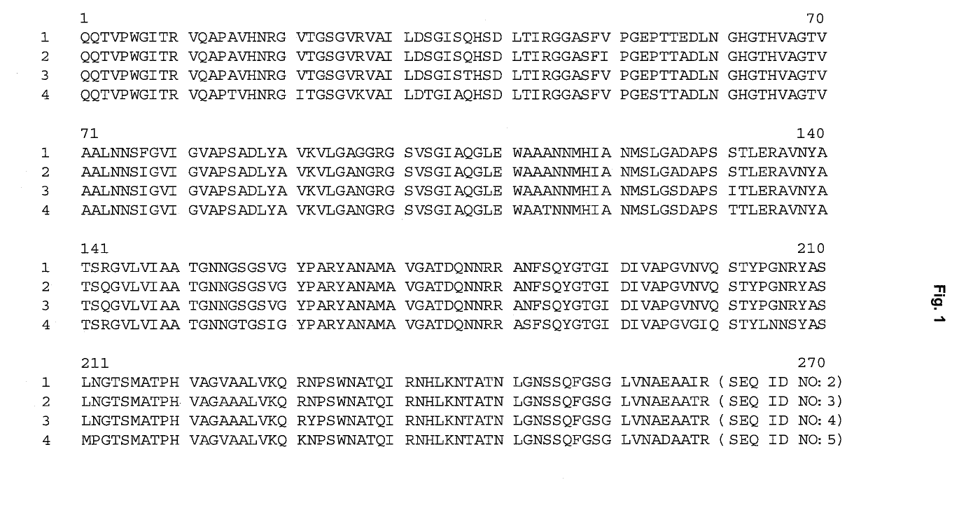

The amino acid sequences of these proteases are also compared with one another in the alignment of FIG. 1.

### Example 3

### Determination of the Washing Power when Used in a Commercial Powdered Laundry Detergent

Textiles, which had been soiled in a standardized manner and obtained from the Eidgenossische Material-Prüfungs- und -Versuchsanstalt, St. Gallen, Switzerland (EMPA) or the Waschereiforschungsanstalt, Krefeld, Germany, were used for this example. The following soilings and textiles were used: A (grass on cotton, EMPA 164), B (milk/oil on cotton PC-10), C (whole egg/carbon black on cotton, 10N), D (chocolate milk/carbon black on cotton, C-03), E (cocoa, EMPA 112) as well as F (blood/milk on cotton, C-5 (044)).

The washing performance of various laundry detergent formulations with this test material was tested. The sample materials were washed for 60 minutes at a temperature of 40° C. The laundry detergent was used at a concentration of 5.9 g per liter wash liquor. Mains water with a hardness of about 16° German hardness was used for washing.

A basic laundry detergent formulation was used as the control laundry detergent and had the following composition (all amounts in weight percent): 10% linear alkylbenzene sulfonate (sodium salt), 1.5% C₁₂-C₁₈ fatty alcohol sulfate (sodium salt), 2.0% C₁₂-C₁₈ fatty alcohol with 7 EO, 20% sodium carbonate, 6.5% sodium hydrogen carbonate, 4.0% amorphous sodium disilicate, 17% sodium carbonate peroxohydrate, 4.0% TAED, 3.0% polyacrylate, 1.0% carboxymethyl cellulose, 1.0% phosphonate, 25% sodium sulfate, remainder: foam inhibitors, optical brightener, fragrances. The following proteases were added at equal activities to the basic laundry detergent formulation for the various test series: Subtilisin HP302 (DE102006022216), Subtilisin TI-1 (WO03/054184), Subtilisin TII-5 (WO03/054185), B. lentus-alkaline protease F 49 (WO 95/23221) and the inventive protease from B. gibsonii.

After washing, the whiteness degree of the washed textiles was measured. The measurement was made with a spectrometer Minolta CM508d, light type D65, 10. The apparatus was calibrated beforehand with a white standard delivered with the apparatus. The results obtained are presented in Table 3 in terms of percent reflectance, i.e. as a percentage in comparison with the white standard, together with the respective starting values. The values are the average of 3 measurements. They allow an immediate conclusion to be drawn on the contribution of the comprised enzyme on the washing performance of the product used.

TABLE 3
Washing results with powdered laundry detergent at 40° C.

| Basic laundry detergent with | A | B | C | D | E | F |
|---|---|---|---|---|---|---|
| Inventive protease from B. gibsonii | 3.6 | 13.8 | 5.3 | 11.4 | 6.2 | 11.3 |
| Subtilisin HP302 | 4.3 | 13.0 | 5.6 | 10.9 | 7.0 | 14.4 |
| Subtilisin TII-5 | 3.6 | 12.5 | 6.5 | 9.5 | 6.8 | 12.3 |
| Subtilisin TI-1 | 4.4 | 12.2 | 5.9 | 9.1 | 4.7 | 12.5 |
| B. lentus alkaline protease F 49 | 1.4 | 10.6 | 6.2 | 2.6 | 5.2 | 5.2 |

The data show that the protease according to the invention from B. gibsonii surpasses the homologous proteases HP302, TII-5 and TI-1 on some stains at 40° C. (stains B and D) and in regard to the other stains affords comparable values to these proteases, and that the protease according to the invention affords better values in regard to all stains, with the exception of stain C, than the established protease B. lentus alkaline protease F49.

### Example 4

### Determination of the Washing Power when Used in a Commercial Liquid Laundry Detergent

The experimental procedure was essentially carried out as described in example 3. The following soilings and textiles were used: A (grass on cotton, EMPA 164), B (milk/oil on cotton PC-10), C (whole egg/carbon black on cotton, 10N), D (chocolate milk/carbon black on cotton, C-03), E (cocoa, EMPA 112) as well as F (blood/milk on cotton, C-5 (044)).

The washing performance of various laundry detergent formulations with this test material was tested. The sample materials were washed for 60 minutes at a temperature of 40° C. The laundry detergent was used at a concentration of 5.9 g per liter wash liquor. Mains water with a hardness of about 16° German hardness was used for washing.

A basic laundry detergent formulation was used as the control laundry detergent and had the following composition (all amounts in weight percent): 0.3-0.5% Xanthane gum, 0.2-0.4% defoamer, 6-7% glycerine, 0.3-0.5% ethanol, 4-7% FAEOS, 24-28% non-ionic surfactants, 1% boric acid, 1-2% sodium citrate (dihydrate), 2-4% soda, 14-16% cocoanut fatty acids, 0.5% HEDP, 0-0.4% PVP, 0-0.05% optical brightener, 0-0.001% colorant, remainder demineralized water. The following proteases were added at equal activities to the basic laundry detergent formulation for the various test series: Subtilisin HP302 (DE102006022216), Subtilisin TI-1 (WO03/054184), Subtilisin TII-5 (WO03/054185), B. lentus alkaline protease F 49 (WO 95/23221) and the inventive protease from B. gibsonii.

After washing, the whiteness degree of the washed textiles was measured. The measurement was made with a spectrometer Minolta CM508d, light type D65, 10. The apparatus was calibrated beforehand with a white standard delivered with the apparatus. The results obtained are presented in Table 4 in terms of percent reflectance, i.e. as a percentage in comparison with the white standard, together with the respective starting values. The values are the average of 3 measurements. They allow an immediate conclusion to be drawn on the contribution of the comprised enzyme on the washing performance of the product used.

TABLE 4
Washing results with liquid laundry detergent at 40° C.

| Basic laundry detergent with | A | B | C | D | E | F |
|---|---|---|---|---|---|---|
| Inventive protease from B. gibsonii | 5.0 | 12.8 | 5.7 | 8.8 | 3.2 | 23.6 |
| Subtilisin HP302 | 2.2 | 10.8 | 3.9 | 2.7 | 3.2 | 13.1 |
| Subtilisin TII-5 | 2.0 | 10.5 | 4.3 | 3.9 | 4.0 | 14.1 |
| Subtilisin TI-1 | 3.2 | 11.2 | 3.1 | 4.6 | 2.4 | 14.2 |

The data show that the protease according to the invention from B. gibsonii in a liquid laundry detergent significantly surpasses to some extent the homologous proteases HP302, TII-5 and TI-1 in regard to all stains at 40° C., with the exception of stain E (cocoa).

What is claimed:

1. An isolated polynucleotide comprising:
(a) the nucleic acid sequence according to SEQ ID NO:1,
(b) the nucleic acid sequence from position 1 to 342 according to SEQ ID NO:1,
(c) the nucleic acid sequence from position 1 to 81 according to SEQ ID NO:1,
(d) the nucleic acid sequence from position 82 to 342 according to SEQ ID NO:1,
(e) the nucleic acid sequence from position 343 to 1152 according to SEQ ID NO:1,
(f) a polynucleotide encoding a polypeptide having the amino acid sequence according to SEQ ID NO:2,
(g) a polynucleotide encoding a polypeptide having the amino acid sequence from position 1 to 114 according to SEQ ID NO:2,
(h) a polynucleotide encoding a polypeptide having the amino acid sequence from position 28 to 114 according to SEQ ID NO:2,
(i) a polynucleotide encoding a polypeptide having the amino acid sequence from position 115 to 383 according to SEQ ID NO:2,
(j) a polynucleotide according to (a) or (e) containing up to 80 mutations,
(k) a polynucleotide according to (b) or (d) containing up to 25 mutations,
(l) a polynucleotide having at least 90% identity to a polynucleotide according to (a) or (e),
(m) a polynucleotide having at least 93% identity to a polynucleotide according to (d),
(n) a polynucleotide capable of hybridizing under stringent conditions with a polynucleotide according to (a) to (i),
(o) a polynucleotide consisting of at least 200 sequential nucleotides of a polynucleotide according to (a), (b), (d), (e), (f), (g), (h) or (i),
(p) a polynucleotide containing deletions and/or insertions and/or inversions of up to 50 nucleotides with respect to a polynucleotide according to (a) to (O), or
(q) a polynucleotide complementary to a polynucleotide according to (a) to (p).

2. The polynucleotide according to claim 1, wherein the polynucleotide encodes a hydrolase.

3. A process for manufacturing a polynucleotide according to claim 1, comprising chemically synthesizing the polynucleotide or synthesizing the polynucleotide by the polymerase chain reaction.

4. A vector comprising the polynucleotide according to claim 1.

5. An isolated polypeptide comprising:
(a) the amino acid sequence according to SEQ ID NO:2,
(b) the amino acid sequence from position 1 to 114 according to SEQ ID NO:2,
(c) the amino acid sequence from position 28 to 114 according to SEQ ID NO:2,
(d) the amino acid sequence from position 115 to 383 according to SEQ ID NO:2,
(e) the amino acid sequence from position 164 to 382 according to SEQ ID NO:2,
(f) the amino acid sequence according to (a) containing up to 14 mutations,
(g) the amino acid sequence according to (b) or (c) containing up to 4 mutations,
(h) the amino acid sequence according to (d) or (e) containing up to 6 mutations,
(i) a polypeptide having at least 96.5% identity to the amino acid sequence according to (a),
j) a polypeptide having at least 97.5% identity to the amino acid sequence according to (d),
k) a polypeptide consisting of at least 81 sequential amino acids of the amino acid sequence according to (a) or (d),
l) a polypeptide consisting of at least 127 sequential amino acids of the amino acid sequence according to (a) or (d), and optionally having one amino acid mutation in the at least 127 sequential amino acids,
m) a polypeptide, consisting of at least 171 sequential amino acids of the amino acid sequence according to (a) or (d), and optionally having up to two amino acid mutations in the at least 171 sequential amino acids,
n) a polypeptide, consisting of at least 192 sequential amino acids of the amino acid sequence according to (a) or (d), and optionally having up to three amino acid mutations in the at least 192 sequential amino acids, or,
o) a polypeptide containing insertions and/or deletions and/or inversions of up to 50 amino acids with respect to the polypeptide according to (a) to (n).

6. The polypeptide according to claim 5, wherein the polypeptide is a hydrolase.

7. A cell comprising the vector according to claim 4.

8. A process for manufacturing a polypeptide, comprising inducing the cell of claim 7 to express the polypeptide encoded by the vector.

9. A composition comprising the polypeptide according to claim 5.

10. The composition according to claim 9, wherein the composition is a laundry detergent or cleaning agent.

11. A process for cleaning textiles or hard surfaces, comprising contacting the textile or hard surface with the laundry detergent or cleaning agent of claim 10 for a period of time sufficient to clean the textile or hard surface.

12. The process of claim 11, wherein the polypeptide is present in a quantity of 40 μg to 96 g.

13. The process of claim 11, wherein the polypeptide is present in a quantity of 50 μg to 72 g.

14. The process of claim 11, wherein the polypeptide is present in a quantity of 100 μg to 48 g.

15. The process of claim 11, wherein the polypeptide is present in a quantity of 200 μg to 24 g.

16. The process of claim 11, wherein the textiles comprise wool or silk.

17. A process for hydrolyzing biofilms on a surface, comprising contacting the surface with the composition of claim 9 for a period of time sufficient to hydrolyze the biofilm.

18. The process of claim 17, wherein the composition is a laundry detergent or cleaning agent.


SEQUENCE LISTING (5 sequences)

SEQ ID NO: 1 - DNA, 1152 bp, Bacillus gibsonii
  CDS (1)..(1152)
atgaaaagaagagtaggaaagcttgtggtagggcttgtatgtgtgacagctctagtaacagtgacagattctgcatctgcagcagaagaaaaggtaaagtacttaataggatttgaagaagaagcagagcttgaagccttcacggaggaagttgaccaaattggtgtattttctgtagaggaacaaagtgtagtagaggatacgttagatattgatgtagacattattgatgaatatgaatatattgatgtactatctgtagaattagatcctgaagatgtagacgcattaagcgatgaagaaggtatttcttttattgaagaagactttgagatgtctatccaacaaactgttccatggggaattactcgtgtacaagctccagctgttcataatcgtggagtaacagggtcgggagtaagagtagctatccttgattcaggtatttctcaacatagtgacttaaccattcgtgggggagcaagctttgtgcctggagagccaacaacggaagatctaaatggtcatggtactcatgttgcgggtacggtagccgcacttaacaattcgtttggtgtaatcggtgttgcgccaagtgcggacttatacgcagtcaaagtactcggtgcaggtggaagaggtagtgttagtggaatcgctcaaggtctagagtgggctgcagcgaataacatgcatattgccaacatgagtctcggcgcagatgcacctagctcaacgcttgagcgtgcagttaactatgcgacaagccgtggtgtattggtcattgcagcgacaggtaacaatggctcaggttcagttggctatcctgctcgttacgcaaacgcaatggctgtaggagcaacggaccagaacaacagacgtgcaaacttttctcaatatggtacaggcattgacatcgtagctcctggggttaatgtacaaagtacgtatccaggaaatcggtatgcgagcctgaatggtacatctatggcaactccacacgtggctggtgtcgctgcgcttgtaaagcaacgtaacccatcttggaatgcaactcaaattcgtaatcatttgaagaatactgcaacaaatctaggaaactcatcgcaatttggtagtggtctagttaatgcagaagcagcaatacgctaa

SEQ ID NO: 2 - protein, 383 aa, Bacillus gibsonii
MKRRVGKLVVGLVCVTALVTVTDSASAAEEKVKYLIGFEEEAELEAFTEEVDQIGVFSVEEQSVVEDTLDIDVDIIDEYEYIDVLSVELDPEDVDALSDEEGISFIEEDFEMSIQQTVPWGITRVQAPAVHNRGVTGSGVRVAILDSGISQHSDLTIRGGASFVPGEPTTEDLNGHGTHVAGTVAALNNSFGVIGVAPSADLYAVKVLGAGGRGSVSGIAQGLEWAAANNMHIANMSLGADAPSSTLERAVNYATSRGVLVIAATGNNGSGSVGYPARYANAMAVGATDQNNRRANFSQYGTGIDIVAPGVNVQSTYPGNRYASLNGTSMATPHVAGVAALVKQRNPSWNATQIRNHLKNTATNLGNSSQFGSGLVNAEAAIR

SEQ ID NO: 3 - protein, 269 aa, Bacillus gibsonii
QQTVPWGITRVQAPAVHNRGVTGSGVRVAILDSGISQHSDLTIRGGASFIPGEPTTADLNGHGTHVAGTVAALNNSIGVIGVAPSADLYAVKVLGANGRGSVSGIAQGLEWAAANNMHIANMSLGADAPSSTLERAVNYATSQGVLVIAATGNNGSGSVGYPARYANAMAVGATDQNNRRANFSQYGTGIDIVAPGVNVQSTYPGNRYASLNGTSMATPHVAGAAALVKQRNPSWNATQIRNHLKNTATNLGNSSQFGSGLVNAEAATR

SEQ ID NO: 4 - protein, 269 aa, Bacillus gibsonii
QQTVPWGITRVQAPAVHNRGVTGSGVRVAILDSGISTHSDLTIRGGASFVPGEPTTADLNGHGTHVAGTVAALNNSIGVIGVAPSADLYAVKVLGANGRGSVSGIAQGLEWAAANNMHIANMSLGSDAPSITLERAVNYATSQGVLVIAATGNNGSGSVGYPARYANAMAVGATDQNNRRANFSQYGTGIDIVAPGVNVQSTYPGNRYASLNGTSMATPHVAGAAALVKQRYPSWNATQIRNHLKNTATNLGNSSQFGSGLVNAEAATR

SEQ ID NO: 5 - protein, 269 aa, Bacillus gibsonii
QQTVPWGITRVQAPTVHNRGITGSGVKVAILDTGIAQHSDLTIRGGASFVPGESTTADLNGHGTHVAGTVAALNNSIGVIGVAPSADLYAVKVLGANGRGSVSGIAQGLEWAATNNMHIANMSLGSDAPSTTLERAVNYATSRGVLVIAATGNNGTGSIGYPARYANAMAVGATDQNNRRASFSQYGTGIDIVAPGVGIQSTYLNNSYASMPGTSMATPHVAGVAALVKQKNPSWNATQIRNHLKNTATNLGNSSQFGSGLVNADAATR